(19) United States
(12) Patent Application Publication
(10) Pub. No.: US 2007/0003525 A1
(43) Pub. Date: Jan. 4, 2007

(54) Hydrogel compositions comprising nucleus pulposus tissue

(76) Inventors: Jeffrey W Moehlenbruck, Austin, TX (US); Chandrashekhar Pathak, Tempe, AZ (US)

Correspondence Address: WILLIAMS, MORGAN & AMERSON, 10333 RICHMOND, SUITE 1100, HOUSTON, TX, 77042, US

(21) Appl. No.: 10/543,931

(22) PCT Filed: Feb. 2, 2004
(86) PCT No.: PCT/US04/03034

§ 371 Date: Jun. 5, 2006

(30) Foreign Application Priority Data
Jan. 31, 2003 (US) 60443978

(51) Int. Cl. A61K 35/30

(52) U.S. Cl. 424/93.7; 424/570

(57) ABSTRACT

Disclosed are methods and compositions useful in the treatment, augmentation and/or repair of soft and/or hard tissues of animals, and in particular, vertebrates such as humans. The invention provides hydrogel compositions for use in the preparation of medicaments for wound healing, cartilage and meniscus repair, dermal augmentation, and bone fusion, as well as methods for the treatment of intervertebral disc impairment. In particular embodiments, the invention provides compositions useful in restoring hydrodynamic function, increasing intervertebral disc height, and improving proliferation and survival of chondrocytes and other cells in intervertebral discs that have been compromised by injury, degenerative disease, congenital abnormalities, and/or the aging process.

The present application claims priority to U.S. Provisional Application Ser. No. 60/443,978, filed Jan. 31, 2003, the entire contents of which is specifically incorporated herein by reference.

### 1. BACKGROUND OF THE INVENTION

1.1 Technical Field

This invention relates generally to methods and compositions useful in the augmentation or repair of soft or hard tissues. More particularly, the invention concerns novel hydrogel compositions for wound healing, cartilage and meniscus repair, dermal augmentation, bone fusion, and treatment of intervertebral disc impairment in humans and other mammals. In one aspect, the compositions are useful in restoring hydrodynamic function, increasing intervertebral disc height, and improving proliferation and survival of chondrocytes and other cells in intervertebral discs that have been compromised by injury, degenerative disease, congenital abnormalities, and/or the aging process.

1.2 Description of Related Art

The human vertebral column (spine) comprises a plurality of articulating bony elements (vertebrae) separated by soft tissue intervertebral discs. The intervertebral discs are flexible joints which provide for flexion, extension, and rotation of the vertebrae relative to one another, thus contributing to the stability and mobility of the spine within the axial skeleton.

The intervertebral disc is comprised of a central, inner portion of soft, amorphous mucoid material known as the nucleus pulposus, which is peripherally surrounded by an annular ring of layers of tough, fibrous material known as the annulus fibrosus. The nucleus pulposus and the annulus fibrosus together are bounded on their upper and lower ends (i.e., cranially and caudally) by vertebral end plates located at the lower and upper ends of adjacent vertebrae. These end plates, which are composed of a thin layer of hyaline cartilage, are directly connected at their peripheries to the lamellae of the inner portions of the annulus fibrosus. The lamellae of the outer portions of the annulus fibrosus connect directly to the bone at the outer edges of the adjacent vertebrae. Thus, the end plates of adjacent vertebrae are coupled to one another by the annulus fibrosus, and in a healthy disc space the two together provide a hydrostatically isolated compartment from which the nucleus pulposus material cannot radially leak or be extruded by mechanical loading of the spine.

The soft, mucoid nucleus pulposus contains chondrocytes, which produce fibrils of collagen (primarily Type II collagen, but also Types IX, XI, and others) and large molecules of negatively charged, sulfated proteoglycans, as depicted in FIG. 1. These non-cellular components of the nucleus pulposus comprise a matrix that allows the cells to proliferate and is essential for a healthy intervertebral disc. Thus, the nucleus pulposus comprises a cellular component, a collagen component, and a proteoglycan component. The term matrix as used herein refers to a composition which provides structural support for, and which facilitates respiration and movement of nutrients and water to and from, an intervertebral disc. The collagenous components of the nucleus pulposus extracellular matrix comprise a scaffold that provides for normal cell (i.e., chondrocyte) attachment and cell proliferation. The negatively charged proteoglycan component of the nucleus pulposus extracellular matrix attracts water to form a hydrogel that envelops the collagen fibrils and chondrocyte cells. In the normal healthy nucleus pulposus, water comprises between 80-90% of the total weight.

The nucleus pulposus thus plays a central role in maintaining normal disc hydrodynamic function. More specifically, the large molecular weight proteoglycans attract water into the nucleus through sieve-like pores in the vertebral end plates. The resulting osmotic pressure within each disc tends to expand it axially (i.e., vertically), driving the adjacent vertebrae further apart. On the other hand, mechanical movements resulting in axial compression, flexion, and rotation of the vertebrae exert forces on the intervertebral discs, which tends to drive water out of the nucleus pulposus. Water movements into and out of an intervertebral disc under the combined influence of osmotic gradients and mechanical forces constitute hydrodynamic functions important for maintaining disc health.

Movement of solutes in the water passing between discs and vertebrae during normal hydrodynamic function facilitates chondrocyte proliferation within the discs by assisting in the respiration and nutrition of the cells. This function is critical to chondrocyte survival since nucleus pulposus tissues of intervertebral discs are avascular (the largest such avascular structures in the human body). Maintaining sufficient water content in the nucleus pulposus is also important for absorbing high mechanical (shock) loads, for resisting herniation of nucleus pulposus matter under such loads, and for hydrating the annulus fibrosus to maintain the flexibility and strength needed for spine stability.

Normal hydrodynamic functions are compromised in degenerative disc disease (DDD). DDD involves deterioration in the structure and function of one or more intervertebral discs and is commonly associated with aging and spinal trauma. Although the etiology of DDD is not well understood, one consistent alteration seen in degenerative discs is an overall decrease in proteoglycan content within the nucleus pulposus and the annulus fibrosus. Because of the hydrophilic properties of proteoglycans, the decrease in proteoglycan content associated with DDD results in a concomitant loss of disc water content. Reduced hydration of disc structures may weaken the annulus fibrosus, predisposing the disc to herniation. Herniation frequently results in extruded nucleus pulposus material impinging on the spinal cord or nerves, causing pain, weakness, and in some cases permanent disability.

Because adequate disc hydration is important for stability and normal mobility of the spine, effective treatment of DDD would ideally restore the disc's natural self-sustaining hydrodynamic function. Such disc regeneration therapy may require substantial restoration of cellular proteoglycan synthesis within the disc to maintain the hydrated extracellular matrix in the nucleus pulposus. Improved hydrodynamic function in such a regenerated disc may result in restoration and reestablishment of intervertebral disc height. It may also provide for improved hydration of the annulus fibrosus, making subsequent herniation less likely.

Prior art approaches to intervertebral disc problems fail to restore normal self-sustaining hydrodynamic function, and thus may not restore normal spinal stability and/or mobility under high loads. One approach to reforming intervertebral discs using a combination of intervertebral disc cells and a bioactive, biodegradable substrate is described in U.S. Pat. No. 5,964,807 to Gan et al., incorporated herein by reference. The biodegradable substrate disclosed in Gan et al., including bioactive glass, polymer foam, and polymer foam coated with sol gel bioactive material, is intended to enhance cell function, cell growth and cell differentiation. Gan et al. describes application of this approach to intervertebral disc reformation in mature New Zealand rabbits, concluding with ingrowth of cells and concurrent degradation of implanted material with little or no inflammation. However, degradation of portions of the implanted material, such as acidic breakdown of PLAs, PGAs and PLGAs, may adversely affect cell growth, cell function and/or cell differentiation.

A somewhat analogous disclosure relating to tissues for grafting describes matrix particulates comprising growth factors that may be seeded with cells; see U.S. Pat. No. 5,800,537 to Bell, incorporated herein by reference. The matrix and cells are applied to scaffolds, which include biodegradable polymers, microparticulates, and collagen which has been cross-linked by exposure to ultraviolet radiation and formed to produce solids of foam, thread, fabric or film. Bell specifically avoids the use of reagents like high salt, or delysidation reagents such as butanol/ether or detergents, which are unfavorably characterized as being responsible for removing from the source tissue factors essential for stimulating repair and remodeling processes. Alternative approaches, in which such factors are obtained from other sources rather than being retained in the tissue, are not addressed.

Still another disclosure related to regeneration of cartilage is found in U.S. Pat. No. 5,837,235 to Mueller et al., incorporated herein by reference. Mueller et al. describes comminuting small particles of autologous omentum or other fatty tissue for use as a carrier, and adding to the carrier growth factors such as transforming growth factor beta (TGF-β) and bone morphogenic protein (BMP). Mueller et al. does not teach cross-linking tissues to create a cross-linked matrix.

Gan et al. is representative of past attempts to restore or regenerate substantially natural hydrodynamic disc function to intervertebral discs, but such techniques have not been proven in clinical trials. Similarly, the approaches of Bell and Mueller et al. have not been widely adapted for disc regeneration, and better approaches are still needed because low back pain sufficient to prevent the patient from working is said to affect 60-85% of all people at some time in their life. In the absence of safer and more efficacious treatment, an estimated 150,000 discectomies and 250,000 spinal fusions are performed each year in the United States alone to treat these conditions. Several prosthetic devices and compositions employing synthetic components have also been proposed for replacement of degenerated discs or portions thereof. See, for example, U.S. Pat. Nos. 4,772,287, 4,904,260, 5,047,055, 5,171,280, 5,171,281, 5,192,326, 5,458,643, 5,514,180, 5,534,028, 5,645,597, 5,674,295, 5,800,549, 5,824,093, 5,922,028, 5,976,186, and 6,022,376.

A portion of the disc prostheses referenced above comprise hydrogels which are intended to facilitate hydrodynamic function similar in some respects to that of healthy natural discs. See, for example, U.S. Pat. No. 6,022,376 (Assell et al.). These prosthetic hydrogels, however, are not renewed through cellular activity within the discs. Thus, any improvement in disc hydrodynamic function would not be self-sustaining and would decline over time with degradation of the prosthetic hydrogel. Healthy intervertebral discs, in contrast, retain their ability to hydrodynamically cushion axial compressive forces in the spine over extended periods because living cells within the discs renew the natural hydrogel (i.e., extracellular matrix) component.

Related U.S. patent application Ser. No. 09/545,441 (filed Apr. 7, 2000) and PCT Intl. Pat. Appl. Publ. No. PCT/US01/11576 (filed Apr. 9, 2001), the entire contents of each of which is specifically incorporated herein by reference in its entirety) discloses compositions and methods for treating DDD comprising nucleus pulposus tissue from a donor vertebrate. The nucleus pulposus tissue is preferably de-cellularized, and even more preferably both de-cellularized and cross-linked. The compositions comprise a moderately viscous fluid matrix that may be delivered, preferably by injection, to the nucleus pulposus of a compromised intervertebral disc. The disclosed matrices not only conform to the available space in the nucleus but also provides a scaffold especially adapted to promote chondrocyte proliferation. Although the disclosed compositions provide a substantial improvement in the treatment of DDD, their use in severely compromised discs may be limited because the matrix may be extruded through fissures or cracks in the annulus fibrosus.

Accordingly, it is an object of the invention to provide improved compositions for treating DDD which are more fully retained in the intervertebral disc space after delivery thereto. It is a further object of the invention to provide improved methods for administering such compositions, including minimally invasive methods. It is a still further object of the invention to provide compositions for treating compromised intervertebral disc that provide improved proliferation and survivability of chondrocytes and other cells in the nucleus pulposus.

In another embodiment it is an object of the invention to provide compositions for treating intervertebral disc compositions in which the viscosity may be controlled. In a further embodiment, the compositions may be delivered to the nucleus pulposus of an intervertebral disc at a first viscosity and thereafter cross-linked or otherwise treated so as to increase the viscosity to a second viscosity greater than the first viscosity. In preferred embodiments, the first viscosity is such as to provide an injectable fluid that may be delivered to the intervertebral disc by, for example, injection, and the second viscosity is such as to yield a semi-solid gel sufficient to avoid extrusion through cracks, holes, or like openings in the intervertebral disc when the disc is subjected to mechanical loading.

### 2. SUMMARY OF THE INVENTION

The present invention comprises methods and compositions for augmentation or repair of soft or hard tissues. Compositions of the invention comprise a three-dimensional fluid matrix derived from soft tissue of a donor animal, and a viscosity control agent. In one embodiment, the soft tissue is decellularized and comprises collagen. In preferred embodiments, the decellularized soft tissue comprises nucleus pulposus tissue from a donor vertebrate. The nucleus pulposus tissue comprises a collagen component and a proteoglycan component. It is also preferred that the nucleus pulposus tissue be stabilized by, for example, crosslinking at least a portion of the collagen component thereof.

The viscosity control agent in may comprise a crosslinkable polymer that can be used to change the viscosity of the matrix in situ after it is delivered to soft or hard tissue of a patient. In particular, the matrix may be delivered to a patient as a relatively low-viscosity liquid and gelled in situ to a much higher viscosity liquid or semi-solid. Matrices of the invention may also comprise a cell enhancement agent to improve cell migration, proliferation, respiration, phenotype retention, and/or survivability in the matrix. In preferred embodiments, the viscosity control agent and the cell enhancement agent may be the same agent.

The three-dimensional fluid matrices of the present invention are biocompatible, substantially non-immunogenic, and resistant to degradation in vivo. Consequently, they can provide important internal structural support for an intervertebral disc undergoing regeneration during a period of accelerated proteoglycan synthesis. The stabilized matrix may be delivered to the intervertebral disc space by injection through a syringe, via a catheter, or other methods known in the art.

Compositions for treating intervertebral disc degeneration comprising a three-dimensional fluid matrix of nucleus pulposus tissue that has been decellularized and crosslinked have been previously disclosed in prior related U.S. patent application Ser. No. 09/545,441 (hereinafter “the '441 application”). It will be appreciated by persons in the art that such cross-linking techniques involve cross-lining of the collagen portion of the nucleus pulposus tissue, and not the proteoglycan components thereof. It has been discovered by the present inventors that compositions disclosed in the '441 application may be enhanced by providing an additional cross-linkable polymer component, which can be cross-linked in situ to control the viscosity of the composition. Thus, the cross-linkable polymer component functions as a viscosity control agent. In preferred embodiments, the viscosity control agent comprises a biocompatible polymer that has been modified by the addition of a cross-linkable moiety that may be cross-linked in situ to increase the viscosity of the composition from a first viscosity to a second, higher viscosity.

It has also been discovered that the compositions of the '441 application may be improved by providing an additional cell enhancement agent to facilitate cell migration, proliferation, phenotype retention and survivability. In preferred embodiments, the cell enhancement agent comprises a proteoglycan, more preferably a proteoglycan modified by the addition of a cross-linkable moiety. Although the cell enhancement agent may comprise a proteoglycan already present in the native nucleus pulposus tissue, it is preferred that the proteoglycan be obtained from a source exogenous to the nucleus pulposus tissue, such as commercially available hyaluronic acid, and then modified by the addition of a cross-linkable moiety.

Compositions of the invention may be delivered to a patient at a first viscosity, and may comprise a viscosity control agent by which the viscosity of the composition may be increased to a second, higher viscosity after delivery to the patient. The compositions may be injectable or otherwise delivered to the patient by minimally invasive means, and may include growth factors, bioactive agents, and/or living cells. The compositions may further comprise a cell enhancement agent to improve the migration, proliferation, extracellular matrix production and survival of living cells.

In particular embodiments, natural disc nucleus pulposus material, obtained from a human or an animal source, is subjected to a photo-activated crosslinking protocol and combined with hydrogel materials which themselves can be cross-linked or polymerized within the intervertebral disc space. The combination of in situ polymerizable hydrogel materials and harvested, natural nucleus pulposus materials produces biocompatible, biodegradable hydrogels useful in augmenting and/or regenerating the nucleus pulposus space in a degenerated disc. The compositions are also useful to treat other conditions such as articular cartilage defects, as a wound healing dressing, and as a carrier for for growth factors or various cells such as intervertebral chondrocytes or stem cells.

Matrices of the present invention may be used alone or in combination with growth factors and/or living cells to facilitate regeneration of the structures of a degenerated intervertebral disc. In patients having sufficient viable endogenous disc cells (chondrocytes) and cell growth factors, the three-dimensional cross-linked matrix alone may substantially contribute to the regeneration of hydrodynamic function in an intervertebral disc in vivo by providing improved mechanical stability of the disc and a more favorable environment for cellular growth and/or metabolism.

While the matrices alone may provide therapeutic benefits, in another embodiment of the invention a combination of a three-dimensional matrix and one or more nucleic acids, protein growth factors, natural or synthetic blood components (such as serum or plasma) or human or animal origin, synthetic mimics of the foregoing, natural or synthetic pain kills, steroidal and non-steroidal anti-inflammatory drugs, anesthetics, antibiotics, or combinations of the foregoing which induce or enhance the efficacy of the matrix. In a preferred embodiment, purified cell growth factors may also be used to treat DDD in discs containing viable chondrocytes in a depleted proteoglycan hydrogel matrix. In this case, the cross-linked collagen component of the nucleus pulposus tissue and the viscosity control agent together provide an expanded remodelable three-dimensional matrix for the existing (native) chondrocytes within the compromised disc, while the cell growth factors induce accelerated proteoglycan production to restore the hydrogel matrix of the patient. The combination of the three-dimensional matrix and one or more purified cell growth factors is referred to as a cell growth medium.

Individual purified cell growth factors may be obtained by recombinant techniques known to those skilled in the art, but a mixture of bone-derived purified cell growth factors suitable for use in matrices of the present invention is disclosed in U.S. Pat. Nos. 5,290,763, 5,371,191 and 5,563,124, all incorporated herein by reference. Bone-derived cell growth factors produced according to these patents are hereinafter referred to as “GFm.”

Cells, including autogenous chondrocytes obtained from the patient and cultured to facilitate growth, as well as exogenous cells of allogenic or xenogenic origin such as intervertebral disc cells, embryonic stem cells, adult pluripotent stem cells, or mesenchymal stem cells, may also be added to matrices of the present invention. The cross-linked collagen and proteoglycan viscosity control agent in compositions of the invention together form a matrix that supports living cells (which may include exogenous cells as well as native disc or other autologous cells) having inherent capability to synthesize Type II collagen fibrils and proteoglycans in vivo, among other extracellular matrix molecules. Where the patient's native disc cells have been removed or are otherwise insufficient to cause such proliferation, living cells may be added to the three-dimensional matrix of cross-linked nucleus pulposus material to further promote disc regeneration.

Accordingly, in another embodiment, the present invention comprises a three-dimensional matrix of decellularized cross-linked nucleus pulposus tissue to which exogenous and/or autologous living cells have been added. The injectable combination of three-dimensional matrix material and exogenous and/or autologous living cells is termed herein an injectable cell matrix. Suitable cells for such an injectable cell matrix may be obtained, for example, from the nucleus pulposus of a mammalian vertebral disc, from cartilage, from fatty tissue, from muscle tissue, from bone marrow, or from bone material (i.e., mesenchymal stem cells), but are not limited to these tissue types. These cells are preferably cultured in vitro to confirm their viability and, optionally, to increase the cells' proliferation and synthesis responses using cell growth factors.

In another aspect, the present invention comprises new hydrogel compositions which will provide closely compatible material properties for nucleus pulposus replacement in the treatment of degenerative disc disease. The use of modified hydrogel chemistries and polymerizable constituents described herein can be used to produce injectable fluid compositions that: (1) are biocompatible and cytocompatible; (2) produce adhesive interaction with host disc tissues (nucleus and annulus) and cohesion for retention in disc and resistance to herniation; (3) have initial viscosity sufficient for delivery via 18-26 gauge needle; (4) can polymerize in situ into a set gel having a desired viscosity within a few minutes; (5) provides restoration of hydration in the disc via establishment of osmotic gradients; (6) provides increased disc height and increased compressive modulus; and (7) ultimately provides effective relief from cervical pain and low back pain.

As used herein, “decellularized” and “decellularization” as used herein refer to tissues and processes by which the native, living cells from the donor tissue are destroyed, fragmented and/or removed. A preferred decellularization approach involves soaking the tissue in a solution having high concentrations of salt (preferably NaCl) and sugar (preferably sucrose). Such high-salt, high-sugar solutions are referred to as HSHS solutions. Other decellularization approaches may be used, however. After the tissues are decellularized and cross-linked, the resulting fluid matrix may be lyophilized for sterilization and storage, and then rehydrated prior to use. Matrices in accordance with the present invention are both resistant to degradation (thereby enhancing durability) and substantially non-immunogenic following implantation into a recipient. It should be emphasized that decellularization in the present invention refers only to the destruction, fragmentation, and/or removal of cells of the donor animal, and not to other cells which may be present in the compositions, such as autogenous cells from patients to whom the compositions are administered, and exogenous stem cells.

“Stabilized” and “stabilization” refer to tissues and processes by which immunogenic, chemical and/or mechanical degradation of the donor tissue after it is implanted into a recipient is reduced or eliminated. A particularly preferred stabilization method is cross-linking (also known as “fixation”), which may be done chemically, thermally, or more preferably by photooxidative catalysts which selectively absorb one or more wavelengths in the visible light or near-visible spectrum. Methods of photo-oxidative crosslinking, referred to hereinafter as “photo-crosslinking,” “photo-linking,” “photofixation” or “photofixing,” are preferred, although other cross-linking processes that retain fundamental material properties of nucleus pulposus tissue may be employed. It is particularly preferred that the cross-linking processes are not cytotoxic.

Photofixation processes are preferred crosslinking methods because in addition to stabilizing the materials against immune response, such processes promote the breakdown and removal of residual cellular elements, provide resistance to proteolytic degradation by enzymes, and reduce immunogenicity of xenogenic and allogenic tissues. In addition, photofixation allows for the retention of many desirable components and processes of the donor nucleus pulposus, including the collagen type II scaffold molecules and hydrophilic proteoglycans.

Photofixation techniques are known in the art and are described in U.S. Pat. Nos. 5,147,514, 5,332,475, 5,817,153, and 5,854,397, all hereby incorporated by reference herein in their entirety. These patents disclose the use of photo-sensitive dyes as the photofixation catalysts. These dyes may include methylene blue, methylene green, rose bengal, riboflavin, proflavin, fluorescein, eosin, and pyridoxal-5-phosphate. Without being limited to any particular theory, it is believed that by absorbing light at particular wavelengths, the photo-sensitive dyes are converted to free radical species which may be used to cross-link amino acids residues, particularly cysteine residues, in the collagens molecules, both intramolecularly and intermolecularly.

In another aspect, natural nucleus pulposus material is combined with synthetic or natural materials that can be polymerized in situ to produce a novel biocompatible, cytocompatible, hydrophilic nucleus replacement material. The material has an excellent capability to promote endogenous or exogenous cell growth, and has the ability to conform to the nucleus space present or created within the intervertebral disc. The materials also can be delivered using minimally invasive surgical instruments and methods. In preferred compositions, the matrix is delivered to the intervertebral disc to be treated at a first viscosity, and cross-linked in situ to provide a semi-solid hydrogel having a second, higher viscosity.

Consistent with the foregoing, in one embodiment, the present invention comprises a fluid matrix for delivery to the nucleus pulposus of an intervertebral disc in need of treatment comprising cross-linked collagen and a cross-linkable viscosity control agent. The cross-linkable viscosity control agent may be cross-linked in situ to provide a matrix for treating DDD, and the matrix may comprise cells, growth factors, drugs such as antibiotics, and other active agents.

In another embodiment, the three-dimensional fluid matrices of the present invention comprise cross-linked, decellularized nucleus pulposus tissue from a donor vertebrate and a viscosity control agent. The donor may be the patient or another animal of the same or different species.

In another embodiment, the compositions of the present invention comprise decellularized nucleus pulposus tissue from a donor vertebrate, wherein at least a portion of the collagen component of the nucleus pulposus tissue has been crosslinked, and further comprising a viscosity control agent by which the viscosity of the composition may be increased from a first viscosity to a second viscosity greater than said first viscosity.

In a further embodiment, the compositions of the present invention comprise a hydrogel for delivery to the nucleus pulposus of an intervertebral disc in need of treatment, said hydrogel comprising a fluid matrix of crosslinked collagen and a crosslinked viscosity control selected from the group consisting of hyaluronic acid, polyalkylene glycols, chitosan, fibrin, and other proteoglycans.

In a different embodiment, the invention provides a matrix for treating intervertebral disc degeneration comprising nucleus pulposus tissue from a donor vertebrate and a viscosity control agent. It is preferred that the nucleus pulposus tissue be decellularized, and even more preferred that the nucleus pulposus comprise a collagen component that is cross-linked.

The viscosity control agent in matrices according to the present invention is preferably cross-linkable in situ by exposure to one or more of ultraviolet, visible, or infrared light. Electromagnetic radiation of other wavelengths may be employed. Preferably, the viscosity control agent is selected from the group consisting of a hyaluronic acid, a polyalkylene glycol, chitosan, fibrin, and other proteoglycans. More preferably, the viscosity control agent comprises a cross-linkable moiety selected from the group consisting of a glycidyl methacrylate moiety, a methacrylate anhydride moiety, and a methacroyl chloride moiety.

In a still further aspect, the present invention provides methods for treating DDD. In a preferred embodiment, the invention comprises a method for treating an intervertebral disc comprising providing a fluid matrix including decellularized nucleus pulposus tissue from a donor vertebrate, wherein at least a portion of the collagen component of said nucleus pulposus tissue is crosslinked, and a cross-linkable viscosity control agent which may comprise a hyaluronic acid, a polyalkylene glycol (including Pluronic polyalkylene glycol and derivatives thereof), chitosan, fibrin, and other proteoglycans. The method further comprises delivering the fluid matrix hydrogel into the nucleus pulposus of the intervertebral disc to be treated, and cross-linking said cross-linkable viscosity control agent by exposure to light.

The method may comprise providing step a matrix having a first viscosity, and crosslinking the matrix to provide a matrix having a second viscosity greater than the first viscosity. In a preferred method, delivery is accomplished by injecting the matrix into the nucleus pulposus of the disc to be treated.

### 3. BRIEF DESCRIPTION OF DRAWINGS

The following drawings form part of the present specification and are included to further demonstrate certain aspects of the present invention. The invention may be better understood by reference to the following description taken in conjunction with the accompanying drawings, in which like reference numerals identify like elements, and in which:

FIG. 1 is a diagram illustrating components of healthy nucleus pulposus tissue in a vertebrate.

FIG. 2 is a diagram illustrating a process for preparation and use of a cross-linked matrix of porcine nucleus pulposus tissue in a preferred embodiment of the invention.

FIG. 3 is a photographic reproduction of an SDS-PAGE (sodium dodecyl sulfate polyacrylamide gel electrophoresis) analysis comparing the amount of proteins extracted from a cross-linked matrix of the present invention with a non-cross-linked control. Lane A shows non-cross-linked control shows substantial protein extraction, and Lane B shows cross-linked matrix demonstrates reduced protein extraction.

FIG. 4 is a photographic comparison of an H & E (hematoxylin and eosin) stained section of fresh porcine nucleus pulposus tissue with a cross-linked matrix of the present invention, both at 300× magnification. The fresh nucleus pulposus shows round, nucleated chondrocytes and intact pericellular matrix “nests,” while the cross-linked matrix shows disrupted, crenated cell fragments, minimal cell membrane material, and further isopropanol sterilization.

FIG. 5 is a photographic reproduction of a stained nitrocellulose membrane comparing the reactivity of Type II collagen digested from a cross-linked matrix of the present invention and a non cross-linked control. Lane A shows pepsin digests of non-cross-linked control react with Type II collagen antibodies. Lane B shows pepsin digests of cross-linked matrix does not react with Type II collagen antibodies.

FIG. 6 is a comparison graph of the hydraulic/swelling capacity of a cross-linked matrix of the present invention and a non-crosslinked control. The cross-linked matrix retains 95% hydraulic capacity.

FIG. 7 is a diagram of an experimental process used to demonstrate stimulation of sheep cell ingrowth, proliferation, and new matrix synthesis in an embodiment of the present invention comprising a cross-linked matrix combined with bone protein growth factors (BP).

FIG. 8 shows the growth factor stimulation of matrix synthesis. The graph shows the results of an Alcian blue assay for matrix production in sheep nucleus pulposus cells stimulated by growth factors. Significant stimulation of matrix production occurred only at μg BP concentrations.

FIG. 9 is a graph indicating the results of immunogenicity tests for a cross-linked matrix of the present invention in rabbit immunizations and sheep serum. Low antibody titers to cross-linked matrix in rabbit immunizations. There were no serum antibodies to cross-linked matrix in vivo (first sheep).

FIG. 10 is a diagram of the protocol for an in vivo study of a matrix and growth factor combination of the present invention.

FIG. 11 is a radiograph of a vertebral column from a sheep sacrificed at 2 months after an injection of a matrix and growth factor combination in an in vivo study of an embodiment of the present invention. Treated and control discs were of normal size and the disc structures appeared normal. The untreated discs showed disjunct endplates, bone resorption and remodeling.

FIG. 12A, FIG. 12B and FIG. 12C are photographic reproductions of histology slides of vertebral discs of a sheep sacrificed at 2 months after an injection of a matrix and growth factor combination of the present invention. FIG. 12A shows untreated disc, FIG. 12BB shows control, and FIG. 12C shows treated disc. After two months post-infection, the untreated disc exhibits extensive degeneration, while the cross-linked matrix/BP treated disc retains normal structures similar to control disc.

FIG. 13 is a radiograph of a vertebral column of a sheep sacrificed at 4 months after an injection of a matrix and growth factor combination in an in vivo study of the present invention. There were no apparent radiographic differences between discs in 4-month sheep.

FIG. 14 is a photographic reproduction of histology slides of vertebral discs of a sheep sacrificed at 4 months after an injection of a matrix and growth factor combination of the present invention. Four months post-injection, untreated disc exhibits degenerative changes, while cross-linked matrix/BP-treated disc is similar to control disc: normal gelatinous nucleus, regular annulus and intact endplates.

FIG. 15A and FIG. 15B are graphs representing the results of an ELISA performed to measure the synthesis of Type II collagen and chondroitin-6-sulfate under growth factor stimulation.

FIG. 16A and FIG. 16B show growth factor stimulation of proteoglycan synthesis in human intervertebral disc nucleus pulposus cells. Shown are graphs (FIG. 16A, 8 day incubation; FIG. 16B, 9 day incubation) indicating the results of an Alcian blue assay for proteoglycan synthesis in human intervertebral disc cells stimulated by growth factor.

FIG. 17 shows growth factor stimulation of proteoglycan synthesis in baboon intervertebral disc nucleus pulposus cells. Shown is a graph depicting the results of an Alcian blue assay for proteoglycan synthesis in baboon intervertebral disc cells stimulated by growth factor.

FIG. 18 is a photograph of a representative matrix according to the present invention comprising photo-crosslinked hyaluronic acid and photo-crosslinked nucleus pulposus material.

FIG. 19 is a photomicrograph of sheep nucleus pulposus cells (SNCs) encapsulated in hydrogels according to the present invention and stained in live/dead stain at time 0.

FIG. 20 is a photomicrograph of sheep nucleus pulposus cells (SNCs) encapsulated in hydrogels according to the present invention and stained in live/dead stain at 24 hours.

FIG. 21 is a photomicrograph of sheep nucleus pulposus cells (SNCs) encapsulated in hydrogels according to the present invention and stained in live/dead stain at 21 days.

FIG. 22, FIG. 23 and FIG. 24 are microphotographs showing the results of studies detailed in Example 24. Normal human articular chondrocytes were encapsulated in a matrix composition and photopolymerized with UV light. Shown are the data obtained at 0 hr incubation.

FIG. 25, FIG. 26 and FIG. 27 are microphotographs showing the results of studies detailed in Example 24. Normal human articular chondrocytes were encapsulated in a matrix composition and photopolymerized with UV light. The data demonstrate that the chondrocytes remained viable and continued to proliferate and migrate across the matrices through 28 days of culture.

FIG. 28 is a graph showing the cytotoxic effects of unpolymerized monomer solution and polymerized hydrogels on sheep nucleus chondrocytes.

FIG. 29 is a graph showing cytotoxic effects of ultraviolet (UV) light of wavelength 365 nm, Irgacure 2959 and free radicals of Irgacure 2959 on sheep nucleus chondrocytes 24 hours post-UV exposure.

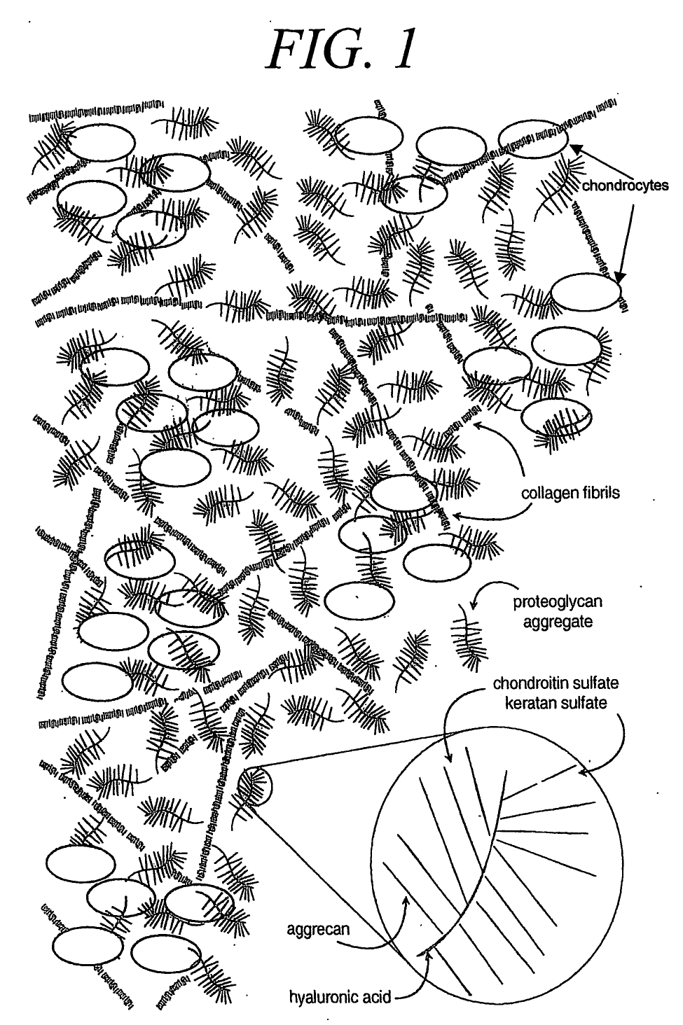

### 4. DESCRIPTION OF ILLUSTRATIVE EMBODIMENTS

Illustrative embodiments of the invention are described below. In the interest of clarity, not all features of an actual implementation are described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure.

While the invention is susceptible to various modifications and alternative forms, specific embodiments thereof have been shown by way of example in the drawings and are herein described in detail. It should be understood, however, that the description herein of specific embodiments is not intended to limit the invention to the particular forms disclosed, but on the contrary, the intention is to cover all modifications, equivalents, and alternatives falling within the spirit and scope of the invention as defined by the appended claims.

Illustrative embodiments of the invention are described below. In the interest of clarity, not all features of an actual implementation are described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure.

Compositions according to the invention comprise a biodegradable fluid matrix to induce and/or enhance regeneration or repair of tissues in the intervertebral disc. In preferred embodiments, the compositions comprise decellularized tissue having a cross-linked collagen component, and a viscosity control agent. The decellularized tissue preferably comprises nucleus pulposus in which at least a portion of the collagen component thereof is cross-linked. The viscosity control agent preferably comprises a proteoglycan derivative, specifically one in which a photo-cross-linkable moiety has been coupled either ionically or covalently to the proteoglycan.

Preferred embodiments of the compositions may be delivered to an intervertebral disc as a semi-viscous liquid at a first viscosity and cross-linked in situ to a semi-solid hydrogel having a second, higher viscosity. The biodegradable matrix comprises hydrophilic molecules, which will maintain and/or increase the “captured” water content in intervertebral disc tissues. The biodegradable matrix may also serve as a carrier substrate for added growth factors and/or appropriate living cell types.

Biodegradable matrices of the present invention furnishes incompressible support when delivered within a closed, secure disc space. Moreover, because it is distributed uniformly within a disc in a first, less viscous state, and then cross-linked in situ, the present fluid matrix has a force distribution effect, hydraulically transmitting forces evenly inside the disc. The matrix thus provides resistance against axial compression and annulus collapse, whereas other matrix materials (for example, polymer sponges and collagen sponges) will rapidly collapse under the axial compressive forces within the disc. The in situ cross-linking process also provides a matrix having enhanced cell proliferation and survivability properties.

In a preferred embodiment, the biodegradable matrix of the present invention is injectable or otherwise deliverable by minimally invasive techniques, significantly reducing both the cost of treatment and the likelihood of complications relative to procedures such as partial discectomy or vertebral fusion. Similarly, the present invention avoids the requirement for boring a hole into the annulus to implant a prosthetic replacement nucleus pulposus device, such as a relatively solid biodegradable matrix, or to evacuate nucleus tissue to create space for an implanted biodegradable substrate.

The matrix of the present invention is a natural material, preferably prepared from normal, healthy nucleus tissue of animals and/or humans and proteoglycan matrix molecules obtained from natural or recombinant sources. Accordingly, the matrix is comprised of proteins and matrix molecules especially adapted for efficient hydrodynamic function in intervertebral discs. It is an important feature of the invention that matrix breakdown products associated with the present invention are digestible by disc cells. In comparison, some matrix materials previously taught (e.g., polyvinyl alcohol) do not break down by physiological processes. In addition, some synthetic polymer substrates create acidic degradation byproducts, in particular PGA and PLA.

Immediate (substantially homogeneous) dispersion of cells within the present matrix is another advantage of the invention. The viscous fluid formulation preferred for injection can be mixed directly with cells of the appropriate type(s) and then delivered immediately to treat an intervertebral disc. In the matrix of the present invention it is not necessary to culture cells and matrix together for some days or weeks before implantation, as it is for certain matrix materials such as PGA and collagen sponges.

The matrix of the present invention is an appropriate substrate for cells, uniquely suited to the ingrowth, proliferation, and residence of intervertebral disc cells. Intervertebral disc cells preferentially grow into and survive in the matrix of the present invention, compared to type I collagen sponges fixed with formalin or glutaraldehyde.

4.1 Illustrative Uses and Applications for Composite Hydrogel Matrix Compositions

The extent of polymerization (i.e., the degree of cross-linking) in the disclosed fluid matrix compositions, and in particular, the composite hydrogel matrix can be adjusted to control the viscosity and adhesive properties of the composition. These matrices may also optionally include one or more various pharmaceuticals, or active agents such as growth factors, antibiotics, analgesics, and the like. These active agents may be included into a matrix, such as a composite hydrogel matrix, in such a fashion as to provide controlled-release, sustained-release, or timed-release of one or more of the active agents into the tissue or repair site over extended time periods. The biocompatible matrices of the invention may also serve as a carrier device for the delivery of cells, such as osteoblasts, chondrocytes, mesenchymal stem cells, etc., to one or more selected tissues in vitro, in vivo, in situ, or ex situ.

The disclosed fluid matrix compositions may find use in a variety of medical, dental, and/or pharmacological applications. For example, the disclosed compositions may be a carrier for growth factor delivery, drug delivery, and gene delivery (delivery of any polymers, peptides, proteins, nucleic acids, etc.).

They may also serve as a carrier for primary cells of any phenotype (fibroblasts, chondrocytes, neurons, mesenchymal stem cells, osteoblasts, etc.) which may be particularly useful in tissue repair or wound healing modalities and regimens. Likewise, the disclosed compositions may be used as a carrier for buffering agents like bicarbonate or other salts—for instance, to offset acidity of degrading polymer scaffolds in a tissue engineering construct or as carrier for nutrients (glucose, serum components, etc.).

In another aspect, the disclosed cross-linkable fluid matrix compositions may find particular utility in tissue augmentation. For example, injectable formulations (gels or solutions) may be used where the desired properties are controlled ranges of density, rigidity, viscosity, and translucence.

4.2 Adjunct Hydrogel Matrix Compositions for Bone Repair

Formulations of the composite hydrogel matrix (i.e., combination hydrogels of GM-HAM and photo-oxidized nucleus pulposus PNP matrix) are suitable for addition to bone repair materials, such as demineralized bone matrix (DBM's) and various bone void fillers (calcium phosphate/collagen constructs or calcium sulfate pellets), for use in the repair of bone defects and non-unions in spine fusion or reconstruction surgery. A combination of composite hydrogel matrix with bone repair materials would produce bone repair compositions with superior handling characteristics for implantation and provide the ability to initiate gelation and retention in situ. When preparing bone repair compositions containing composite hydrogel matrix, mixtures should provide sufficient density of reactive groups in GM-HAM to enable the polymerization chemistry to proceed under UV light. Final formulations may be somewhat soft and gelatinous, or rigid, depending on the ratio of composite hydrogel matrix to bone repair materials. For example, the composite hydrogel matrix could be combined with Ca₂SO₄ pellets to produce a thick viscous paste, which would then be injected into a bone void space and UV-polymerized in situ to retain the material in place. An illustrative calcium phosphate collagen construct for use in bone repair is described in U.S. Patent Appl. Publ. No. US2002/0114795 (specifically incorporated herein by reference in its entirety).

4.3 Hydrogel Matrix Compositions for Use in Dressings and for the Healing and Repair of Wounds, Burns, and Other Soft-Tissue Injuries

Formulations of the composite hydrogel matrix (including, for example, a combination hydrogel that comprises GM-HAM and photo-oxidized nucleus pulposus PNP matrix) are also suitable as wound dressing materials. The composite hydrogel matrix provides adhesive properties which are desirable in wound dressing compositions. Pharmaceutically-acceptable formulations of one or more of the disclosed composite hydrogel matrices maintain adhesion to skin of varying moisture levels, and thus make them ideally suited to keeping its position on the skin. Such composite hydrogel matrices can be applied to a wound as a viscous gel and UV-polymerized in place. The polymerized composite hydrogel matrix may serve to maintain a sterile antimicrobial barrier over the wound area, while simultaneously permitting air and moisture exchange, and skin exudates to be transmitted away from the skin.

4.4 Hydrogel Matrix Compositions for Use in Articular Cartilage Repair

Formulations of one or more of the disclosed composite hydrogel matrices (e.g., combination hydrogels of GM-HAM and photo-oxidized nucleus pulposus PNP matrix) are also suitable for use in articular cartilage repair. The composite hydrogel matrix compositions can also be used as a stand-alone implantable material which is injected as a viscous fluid paste into a prepared articular cartilage defect site and UV-polymerized in place. The fluid paste of composite hydrogel matrix would flow into and fill in all areas within the cartilage defect before polymerization, thus forming a tight seal and uniform interface at the host cartilage: implant junction. Features of the composite hydrogel matrix material provide for its adherence to the underlying subchondral bone layer and to the rim walls of cartilage which demarcate the defect site. The host cartilage would integrate with the composite hydrogel matrix and gradually remodel the matrix into de novo articular cartilage. Several possible approaches could incorporate the composite hydrogel matrix into devices to repair articular cartilage: use as an adjunct gel in the micro-fracture technique, combination with other scaffold materials to form cartilage plugs, and use as a cell-seeded carrier device for delivery of autologous chondrocytes or mesenchymal stem cells, etc.

For use in repair of articular cartilage via the micro-fracture technique, the articular cartilage would be debrided to remove damaged cartilage and prepared to create a defect with defined borders or walls. The surface in the defect may be shaved just down, but not through, the subchondral bone layer. A micro-picking tool may then be used to create evenly spaced fractures through the subchondral bone. This micro-fracture technique causes seepage of underlying bone marrow and stem cell-rich blood into the prepared defect site. At such time, the composite hydrogel matrix is added to the slowly clotting mixture of blood, fibrin, and stem cells which has filled the cartilage defect. The UV-polymerization of the composite hydrogel matrix bone marrow/stem cell mixture allows for the retention of these repair elements within the articular cartilage defect site.

A second approach involves inclusion of the composite hydrogel matrix into a formed scaffold such as a collagen sponge. For example, the composite hydrogel matrix gel may be applied onto the sponge construct in order to soak and permeate the collagen sponge. After thorough permeation of the gel into the collagen sponge, the gel-collagen sponge would be then set into a prepared cartilage defect. The composite hydrogel matrix gel material would provide enhanced surface adhesion to the cartilage and UV-polymerization of the composite hydrogel matrix gel in place would provide enhanced retention of the sponge in the defect. The polymerized composite hydrogel matrix gel would form a smooth, lubricious layer on the exposed surface of the construct and provide a superior interface for host: sponge construct integration within the cartilage defect. In this approach, the composite hydrogel matrix gel could be loaded with growth factors which are chondrogenic in nature, or alternatively, the gel could be loaded with specific cell types (chondrocytes, stem cells) which would populate the gel-collagen sponge construct and then begin forming cartilage matrix to eventually remodel the sponge construct.

A third approach involves the combination of the composite hydrogel matrix gel with cell therapies for articular cartilage repair. It is proposed that current cell therapy approaches (e.g., Genzyme Carticel) would be substantially improved by the addition of the composite hydrogel matrix for delivery of the chondrocytes. In this approach, harvested autologous chondrocytes, allograft chondrocytes, or mesenchymal stem cells could be encapsulated within the gel material, injected into the articular cartilage defect, and UV-polymerized in place. The advantages described above for host integration and space filling would be expected to provide a superior result in outcomes for repair of articular cartilage.

### 5. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### 5.1 Example 1

### Preparation of a Cross-Linked, Fluid Matrix Suitable for Treatment of Degenerative Disc Disease

A three-dimensional fluid matrix of cross-linked nucleus pulposus tissue in accordance with an embodiment of the present invention may be prepared from donor vertebrates. Although porcine donors were used in a particularly preferred embodiment, nucleus pulposus tissues from other vertebrates may also be used, although mammalian vertebrates are preferred (e.g., human, porcine, bovine, ovine, etc.).

Although nucleus pulposus tissues may be harvested by a variety of methods from many vertebral donors, in a preferred embodiment nucleus pulposus tissues were dissected aseptically from spinal intervertebral discs of pigs. In a sterile environment (i.e., a laminar flow hood), the annulus fibrosus of porcine donors was sliced radially and the vertebral end plates separated to expose the nucleus pulposus. The latter material was curetted out of the central portion of the disc, devoid of annulus and end plate tissues.

The nucleus pulposus tissues thus harvested were inserted into sterile dialysis (filter) tubing having a preferred molecular weight cutoff of about 3500 Daltons to substantially prevent loss of low molecular weight proteoglycans from the tissues while substantially reducing bacterial or other contamination. Other semipermeable membranes or filtering membrane types may be used to perform these functions.

The nucleus pulposus tissues to be cross-linked are also preferably treated to destroy and remove donor cells and/or cell fragments. To this end, dialysis tubing containing nucleus pulposus tissue was submerged in a high-salt, high-sucrose (HSHS) solution of approximately 2.2%: 8.4% wt./vol. (respectively) for about 48 hours. Concentration ranges for the HSHS solution may be from 1% to 50%, but a preferred HSHS solution contains 220 grams NaCl and 837.5 grams of sucrose in 10 L water. Preferred HSHS incubation times are from about 24 to approximately 72 hours, although shorter or longer times may also advantageously be used. Exposure to this HSHS solution results in osmotic destruction and fragmentation of native chondrocyte cells (decellularization), and further results in denaturation of soluble cellular proteins and nucleic acids. The HSHS solution may also contain other reagents which further degrade nucleic acids (including but not limited to sulfones and nucleases), and other reagents which can extract membrane lipids (including but not limited to alcohol, chloroform, and methanol). Although native cells of the donor may be retained in other embodiments of the invention, decellularization and denaturation are preferred where exogenous (particularly xenogenic) tissues are used, so as to reduce the potential for immunogenic responses. Processes other than exposure to HSHS solutions may be used for his purpose.

Cross-linking of the nucleus pulposus tissues is preferably accomplished by a photo-mediated process in accordance with U.S. Pat. Nos. 5,147,514, 5,332,475, 5,817,153, and/or 5,854,397 (each of which is specifically incorporated herein by reference in its entirety). In one such process, a photoactive dye (methylene blue) was dissolved in the HSHS solution at a preferred dye concentration of about 20 mg/liter. The photoactive dye was allowed to permeate the nucleus tissues within the dialysis tubing during the initial storage/decellularization process in HSHS. A wide range of photoactive dyes and concentrations, as taught in the foregoing patents, may be used to obtain cross-linked fluid matrices suitable for use in regenerating mammalian disc tissues. Preferred dyes include methylene blue and methylene green at concentrations of about 0.001% to about 1.0% wt./vol.

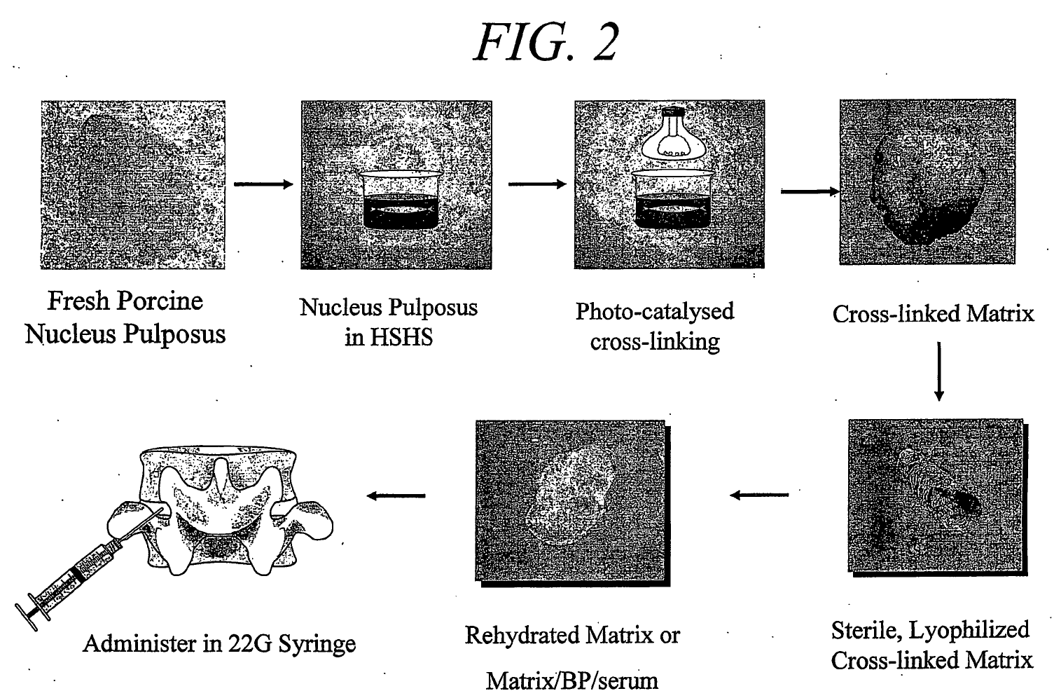

To cross-link the collagen within the nucleus tissues, the dialysis tubing containing the dye-permeated nucleus tissues was placed in a photooxidation chamber and exposed to broad-spectrum visible light for 48 hours. In preferred embodiments of the invention, the tissues may be cross-linked from about 24 to about 72 hours. A solution of methylene blue in phosphate buffered saline (“PBS”) was maintained under controlled temperature at 10° C. and circulated around the dialysis tubing within the photooxidation chamber to provide substantially constant temperature regulation of the nucleus tissues. Precise temperature control is not critical to the practice of the invention; however, maintaining a relatively cooler temperature is preferred to avoid damaging the tissues. Following photo-crosslinking of the collagen, the treated nucleus tissues were collected, lyophilized in a vacuum under centrifugation, and finely pulverized in a freezer-mill under liquid nitrogen. The cross-linked matrix product thus prepared can be sterilized using gamma radiation, ethylene oxide (or other sterilants) and stored at −80° C. until rehydrated for use. A preferred process for preparing a matrix according to the present invention is illustrated in FIG. 2.

In addition to preparation of the cross-linked matrix, control (non-crosslinked) tissues were prepared following the above procedures, except that they were not exposed to light. These control, non-crosslinked tissues were used for comparison purposes.

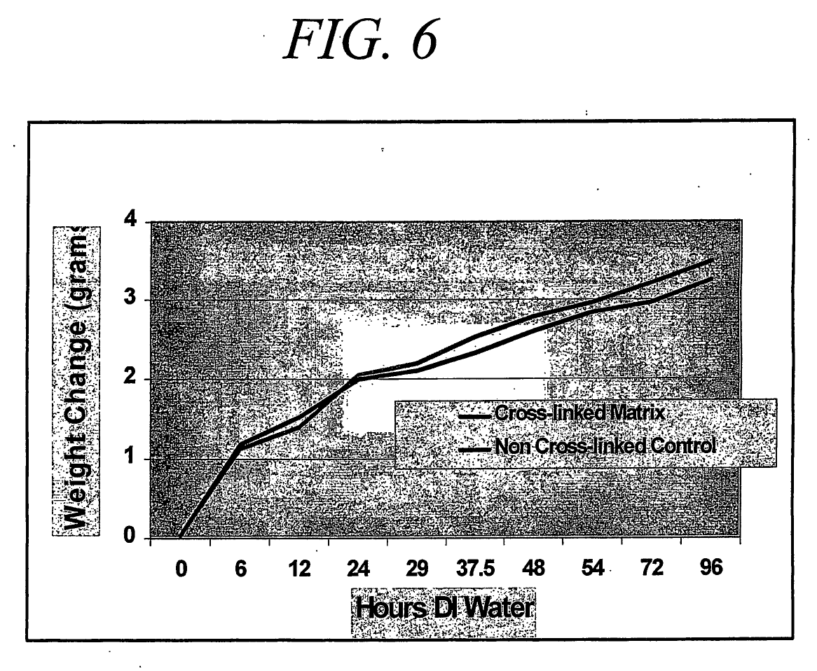

To investigate the swelling capacity of cross-linked matrix versus non-crosslinked control, lyophilized samples of cross-linked matrix and non-crosslinked control were suspended in water and the increase in weight due to water absorption was measured at various times from 0 to 96 hours. As illustrated in FIG. 6, the cross-linked matrix retained 95% of the hydraulic capacity of the non-crosslinked control.

### 5.2 Example 2

### Testing of Fluid Matrix to Evaluate Protein Modification Induced by the Cross-Linking Process

One half gram of the matrix material obtained prior to the lyophilization step of Example 1 was placed in 15 ml of a solution of 4 M guanidine hydrochloride and agitated on a shaker for 24 hours to solubilize proteoglycans. After centrifugation, the supernatant was discarded and the pellet washed in distilled water 3 times for 5 minutes each. The pelleted matrix material was then removed and blot-dried on filter paper.

One hundred mg of the blot-dried matrix was placed in a 1.5 ml microcentrifuge tube with 1000 μl of 1% sodium dodecyl sulfate (SDS) containing 5% β-mercaptoethanol (BME). The matrix in SDS/BME was boiled for one hour to extract proteins (e.g., collagens). Samples were then centrifuged at 12000 rpm for 1 hour and aliquots of the supernatant were subjected to electrophoresis in gradient polyacrylamide gels.

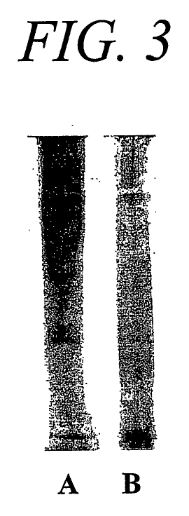

Gels were stained with Coomassie blue or silver to visualize proteins extracted by the SDS/BME and heat treatment. As illustrated in FIG. 3, collagen bands stained prominently in control, non-crosslinked tissues but exhibited only faint staining in cross-linked matrix. These results demonstrated that in the cross-linked matrix material, collagen proteins were not easily extracted by the above treatment, indicating that crosslinking had occurred. In contrast, stained gels of the control tissues demonstrated that collagen proteins were readily extracted from non-crosslinked material by the above treatment. See FIG. 3.

### 5.3 Example 3

### Matrix Histology to Evaluate Cellular Debris and Residual Membranous Material

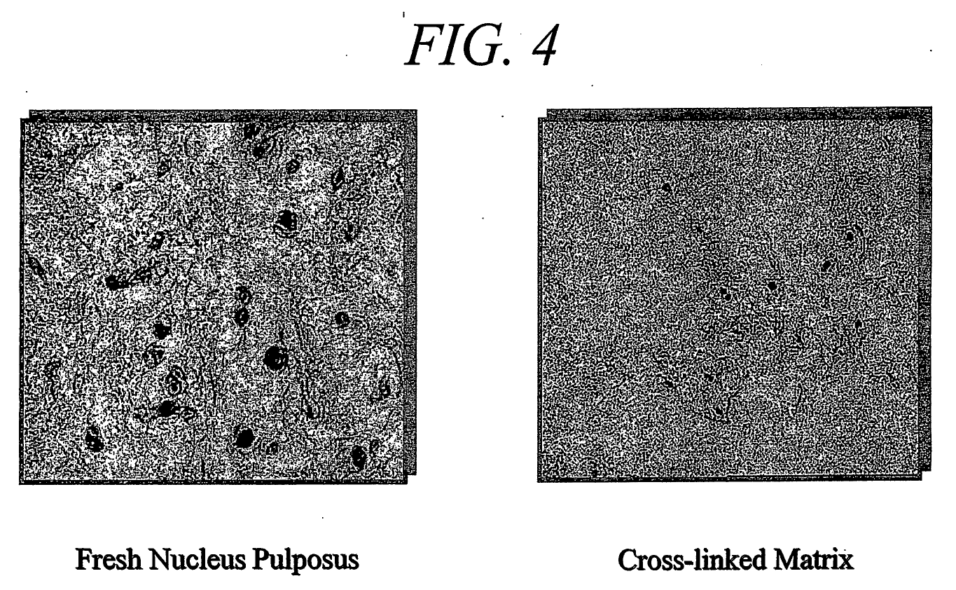

Cross-linked matrix material obtained prior to the lyophilization step of Example 1 was placed in 4% paraformaldehyde for tissue fixation. Standard histology techniques of embedding, sectioning, and staining of sections with hematoxylin & eosin dyes were performed. Visualization of cross-linked matrix in H & E-stained sections demonstrated that the matrix preparation process facilitates destruction of cellular membranes and intracellular elements, with minimal membrane material remaining as compared to fresh porcine nucleus pulposus material as well as non-crosslinked tissue decellularized by HSHS treatment, freeze-thaw cycles, and HSHS treatment plus freeze-thaw cycles. These data are illustrated in FIG. 4.

### 5.4 Example 4

### Evaluation of Matrix Antigenic Reactivity Using Monoclonal Antibodies to Type II Collagen

Cross-linked matrix material obtained prior to the lyophilization step of Example 1 was also subjected to pepsin digestion to cleave Type II collagen proteins. The protein digests were run on SDS/PAGE and then transferred to a nitrocellulose membrane. Total protein transferred to the membrane was visualized using colloidal gold.

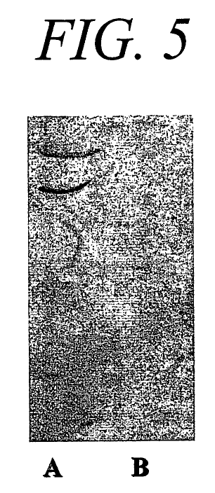

The visualized nitrocellulose membranes were incubated with a mouse monoclonal antibody to Type II collagen and a secondary antibody (anti-mouse) conjugated with alkaline phosphatase. The antibody reactivity was visualized through addition of alkaline phosphatase substrate. As depicted in FIG. 5, the antibodies toward Type II collagen did not react with pepsin digests of the cross-linked matrix as much as with the pepsin digests of the non-crosslinked control tissue. The results indicate that the matrix of the invention may have reduced antigenic epitopes for Type II collagen, and thus have less immunogenicity than non-crosslinked tissues. These results are illustrated in FIG. 5.

### 5.5 Example 5

### Evaluation of Matrix Immunogenicity in Rabbit Antisera Production

One gram of the lyophilized and pulverized matrix material prepared according to Example 1 was dispersed in PBS (i.e., rehydrated) and centrifuged. The protein concentration of the supernatant was then determined using the BCA assay and the supernatant was diluted with PBS to a final concentration of 200 μg of protein per ml of PBS. The diluted supernatant was then sterilized for injection protocols. Three rabbits were immunized with 100 μg of protein from the sterilized supernatant. Each rabbit received nine immunizations over a 14 week period and sera was collected from the rabbits on a regular schedule.

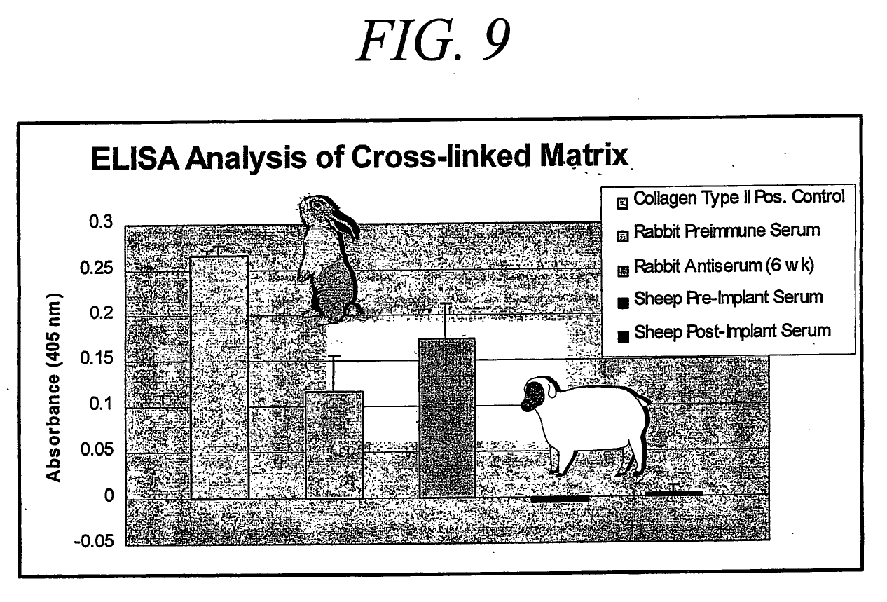

Antisera production against the protein extract was measured using an enzyme-linked immunosorbent assay (ELISA). Type II collagen was included as a positive control in the ELISA. Colorimetric evaluation of antiserum directed against the matrix material demonstrated very low immunogenicity in rabbits. These results are illustrated in FIG. 9.

### 5.6 Example 6

### Matrix Formulation Including Serum and Other Fluids for Injections and Delivery

One gram of the lyophilized and pulverized matrix material prepared according to Example 1 was sterilized with 70% ethanol and the ethanol was removed by successive PBS rinses. The dispersed matrix was centrifuged and the pellet was suspended in heat-inactivated sheep serum at a ratio of 0.5 g lyophilized matrix to 1 ml serum to prepare a viscous fluid matrix which can be loaded into a standard syringe and delivered via a small gauge needle. In preferred embodiments of the invention, the serum is collected from the vertebrate animal or human patient to be treated, heat-inactivated to destroy unwanted protein components (complement proteins), and passed through a 0.2 micron sterile filtration unit. Different matrix/serum ratios may also be advantageously employed. Ratios ranging from 0.1 g to 2.0 g of lyophilized matrix to 1 ml of serum are preferred.

Serum is a preferred fluid for mixture and delivery of the cross-linked matrix of the present invention because it contains various intrinsic growth factors that are beneficial to intervertebral disc cells. Serum also serves as a suitable carrier for extrinsic protein growth factors and/or small molecules. The beneficial effects of extrinsic growth factors on intervertebral disc cells are enhanced by the addition of serum.

Other fluids are also suitable for mixture and delivery of the viscous fluid matrix. For example, sterile saline or sterile water may also be used. The examples herein are not meant to be limiting as to the variety of carrier fluids that may be used to mix and deliver the matrix in the present invention.

### 5.7 Example 7

### Injection of Matrix Formulation to Intervertebral Discs Using Pressure-Mediated Syringe

Matrix material was prepared according to Example 6 (mixed with serum) to form a viscous fluid and loaded into a standard syringe having a small gauge needle (e.g., 18-31 gauge) attached. Syringe injection pressure can be controlled simply by the fingers of the hand. In other embodiments of the invention, pressure to inject the viscous fluid can be controlled by an external device which concomitantly measures (e.g., via a pressure transducer) and delivers (e.g., by compressed air) a predetermined force to the syringe plunger.

In one preferred embodiment of this device, a thermal element is included in the needle. By providing a needle having a thermal element, it is possible to deliver heat to the outer layers of the annulus fibrosus at the end of the treatment and during removal of the syringe needle in order to shrink collagen fibers around the needle and effectively seal the site of needle penetration.

It is further contemplated that the matrix of the present invention can be delivered to the disc space of a patient transpedicularly (i.e., through the pedicle of the vertebrae). In particular, the cross-linked matrix can be administered percutaneously via a biopsy cannula inserted through a channel in the pedicle. After delivery of the matrix, the channel can then be filled with bone cement or other like material to seal the channel.

### 5.8 Example 8

### Isolation of Human, Sheep, and Baboon Intervertebral Disc Nucleus Pulposus Cells

Human intervertebral nucleus pulposus tissues were collected during surgery, suspended in Dulbecco's Modified Eagle Medium/Nutrient Mixture F-12 (DMEM/F-12) in a 1:1 vol./vol. mixture supplemented with antibiotics. The tissues were kept on ice until dissection, at which time they were rinsed 2-3 times in sterile Dulbecco's Phosphate Buffer Saline (DPBS) to remove any blood. In a laminar flow hood, the nucleus tissues were isolated and diced into small (2 mm) cubes, and then placed in tissue culture medium (hereinafter referred to as “TCM”) comprising DMEM/F-12 culture media supplemented with 10% heat inactivated fetal bovine serum, 0.25% penicillin, 0.4% streptomycin, 0.001% amphotericin B, and 50 μg/ml ascorbic acid. Only tissues clear of blood and other anomalous elements were used. Placed on a shaker at 37° C., the tissues were digested with 0.01% hyaluronidase (Calbiochem) in TCM for 2 hours, 0.01% protease (Sigma) in TCM for 1 hour, and 0.1% collagenase Type II (Sigma) in TCM overnight to obtain a suspension of human intervertebral disc nucleus pulposus cells.

The foregoing procedure was also applied to sheep and baboon intervertebral disc nucleus pulposus tissues to obtain suspensions of sheep and baboon intervertebral disc nucleus pulposus cells, respectively.

### 5.9 Example 9

### Primary Culture and Expansion of Human, Sheep, and Baboon Intervertebral Disc Nucleus Pulposus Cells

Human intervertebral disc nucleus pulposus cells from Example 8 were expanded by culturing in TCM at 37° C. in 5% CO₂ atmosphere and 95% relative humidity. The TCM was changed every 2-3 days and the cells were passaged with trypsin to another container, when 80-90% confluent, for continued expansion.

The foregoing procedure was also applied to sheep and baboon intervertebral disc nucleus pulposus tissues to obtain an expanded supply of sheep and baboon intervertebral disc nucleus pulposus cells.

### 5.10 Example 10

### Alcian Blue Assay of Disc Cell Matrix Production in Human, Sheep, and Baboon Intervertebral Disc Nucleus Pulposus Cells

Human intervertebral disc cells from Example 9 were seeded and grown in 24 well plates in TCM in the presence or absence of exogenous growth factors. At various time points, TCM was aspirated out from the wells and the wells washed 3 times with PBS. The cells were then fixed with 4% paraformaldehyde (pH 7.4) for 10 min. The fixed cells were washed 2 times with PBS and then stained overnight with 0.5% Alcian blue in 0.1N hydrochloric acid (pH 1.5). After overnight staining, excess stain was rinsed out with 3 rinses of PBS. The remaining Alcian blue stain (bound to proteoglycans) was dissolved overnight into 6 M guanidine hydrochloride and the absorbance at 630 nm was measured using a spectrophotometer, providing an indication of the induction of matrix production by exogenous growth factors in human nucleus pulposus cells.

The foregoing procedure was also applied to sheep and baboon intervertebral nucleus pulposus cells from Example 9 to obtain an indication of the induction of matrix production by exogenous growth factors in sheep and baboon nucleus pulposus cells.

### 5.11 Example 11

### Enzyme Linked Immunosorbent Assay (Elisa) on Ovine Intervertebral Disc Nucleus Pulposus Cells

To detect specific antigenic epitopes in the synthesized matrix, sheep intervertebral nucleus pulposus cells from Example 9, seeded and grown in monolayer, were fixed in 2% glutaraldehyde for 1 hour at room temperature. The fixed cells were washed 3 times with TBS for 5 min. each. To block non-specific antibody binding, the cells were incubated in a solution of Tris buffered saline (TBS) containing 1 mM ethylene-diamine-tetraacetic acid (EDTA), 0.05% Tween-20™, and 0.25% bovine serum albumin for 1 hour. The blocking step was followed by 3 washes with TBS for 5 min. each. The cells were incubated with the primary antibody at room temperature for 2.5 hours, and the excess primary antibody was removed by 3 washes with TBS for 5 min. each. A second incubation with blocking buffer was performed for 10 min., followed by 3 washes with TBS. The cells were then incubated with the secondary antibody, which was conjugated with alkaline phosphatase enzyme, for 3 hours at room temperature. The unbound secondary antibodies were removed by 3 washes of TBS for 5 min each. The bound primary and secondary antibodies were detected by addition of an enzyme-specific substrate which produced a colored reaction. The colorimetric measurement was performed using a spectrophotometer, providing a quantitative measure of the presence of the bound antibodies.

### 5.12 Example 12

### Effect of Exogenous Growth Factors on Proteoglycan Synthesis in Ovine Intervertebral Disc Nucleus Pulposus Cells

Transforming growth factor-β1 (TGF-β1) and a mixture of bone-derived protein growth factors (BP) produced according to U.S. Pat. Nos. 5,290,763, 5,371,191 and 5,563,124, were tested for their effects on stimulation of proteoglycan synthesis in ovine nucleus pulposus cells. Sheep intervertebral disc nucleus cells were collected and cultured as described in Examples 8 and 9. Sheep cells were seeded in micromass (200,000) into the wells of a 24-well plate. Growth factor dilutions were prepared in TCM supplemented with 0.5% heat-inactivated fetal bovine serum. TGF-β1 and BP were both tested at 10 ng/ml; BP was also tested at a concentration of 10 μg/ml. Control wells without growth factors contained TCM supplemented with 0.5% and 10% heat-inactivated fetal bovine serum. The cells were incubated in continuous exposure to the various growth factors for 7 and 10 days. At these time points, the cells were fixed and the amount of proteoglycan synthesis was measured by the Alcian blue assay as described in Example 10.

At both 7 and 10 day time points, proteoglycan synthesis was significantly greater in the 10% fetal bovine serum control cultures than in the 0.5% fetal bovine serum control cultures. At the 7 day time point, BP at the higher 10 μg/ml concentration produced a significant (93%) increase in proteoglycan synthesis above the level in 10% serum control culture and a greater (197%) increase above the 0.5% serum control. Slight increases in proteoglycan synthesis above the 0.5% serum control were observed in the 10 ng/ml TGF-β1 and BP cultures, but these increases were not significant.

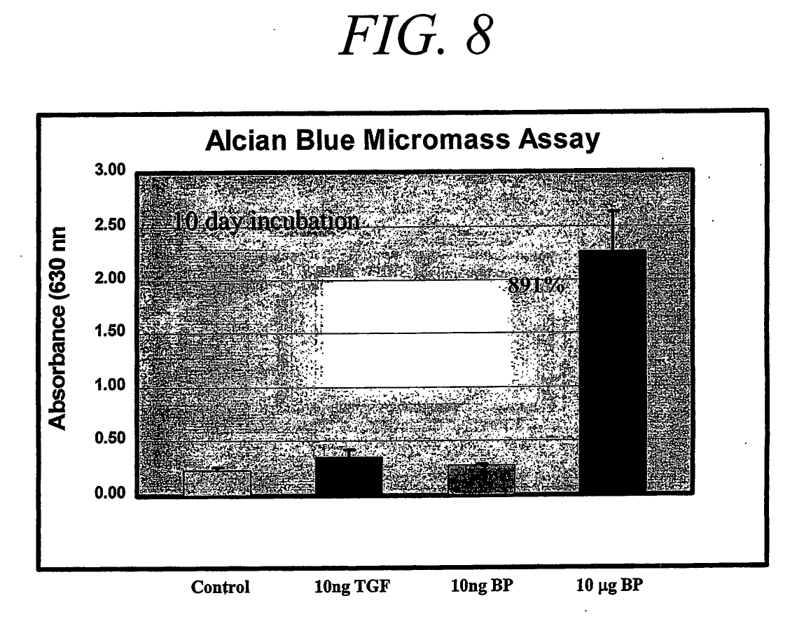

At the 10 day time point (FIG. 8), 10 μg/ml BP produced a significant increase (132%) in proteoglycan synthesis over the 10% serum control, while 10 ng/ml TGF-β1 produced a significant increase (52%) above the 0.5% serum control. At 10 ng/ml, BP exhibited a modest 20% increase in proteoglycan synthesis over the 0.5% serum control, while at the 10 μg/ml concentration, BP produced an 890% increase above the 0.5% serum control.

### 5.13 Example 13

### Effect of Exogenous Growth Factors on Type II Collagen and Chondroitin-6-Sulfate Produced by Ovine Intervertebral Disc Nucleus Pulposus Cells

TGF-β1 and BP were tested for their effects on stimulation of Type II collagen and chondroitin-6-sulfate synthesis in sheep intervertebral disc nucleus pulposus cells. The cells were obtained and cultured as described in Examples 8 and 9 and seeded into tissue culture dishes. The TGF-β1 and BP growth factors were prepared in TCM supplemented with 0.5% heat-inactivated fetal bovine serum. TGF-β1 was tested at a concentration of 10 ng/ml; BP was tested at a concentration of 10 μg/ml. Control cultures were incubated in TCM supplemented with 0.5% serum alone.

After incubation with growth factors for 7 days, cell cultures were fixed in 2% glutaraldehyde and the quantity of Type II collagen and chondroitin-6-sulfate produced in the cell cultures was detected by ELISA according to the procedure described in Example 11. The primary antibodies used were mouse anti-human Type II collagen and mouse anti-human chondroitin-6-sulfate.

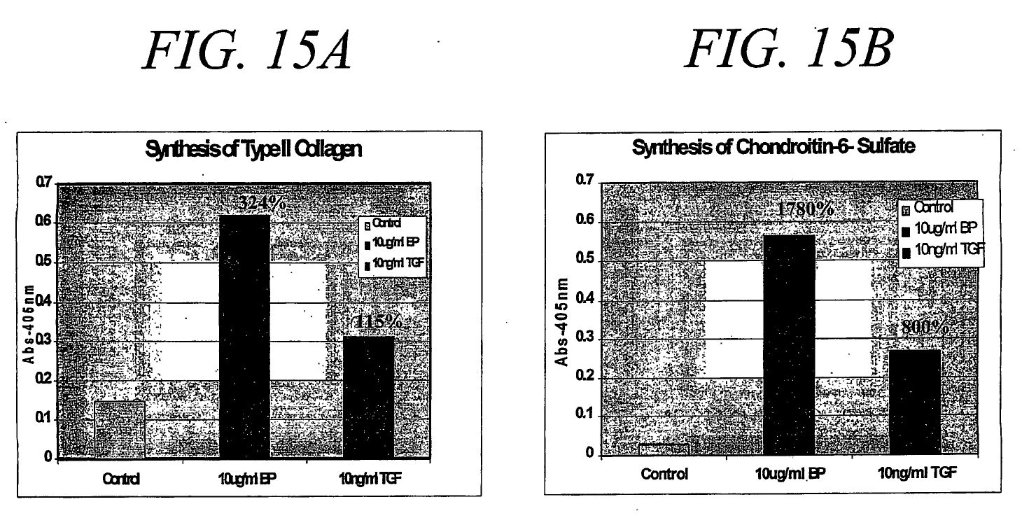

At 7 days, cell cultures incubated with 10 μg/ml BP produced 324% more Type II collagen and 1780% more chondroitin-6-sulfate than control cultures. Ten (10) ng/ml TGF-β1 increased production of Type II collagen by 115% and chondroitin-6-sulfate by 800% over controls. See FIG. 15.

### 5.14 Example 14

### Effect of Exogenous Growth Factors on Proteoglycan Synthesis in Human Intervertebral Disc Nucleus Pulposus Cells

TGF-β1 and BP were tested for their effects on stimulation of proteoglycan synthesis in human nucleus cells. Human intervertebral disc nucleus pulposus cells obtained from Disc L5-S1 of a 40 year old female patient were cultured as described in Examples 8 and 9 and seeded into 24-well plates. After the cells adhered to the well surface, multiple dilutions of different growth factors were added. The concentrations of growth factors tested were 10 ng/ml TGF-β1, and 10 and 20 μg/ml of BP. The dilutions were prepared in TCM. The cells were fixed after 5 and 8 days of continuous exposure to growth factors and proteoglycans synthesized were detected by the Alcian blue assay as described in Example 10.

At 5 days only BP produced a significant increase in Alcian blue staining over controls. At 10 μg/ml BP there was a 34% increase over the control while at 20 μg/ml there was a 23% increase over the control. The difference between the averages of 10 and 20 μg/ml BP was not significant.

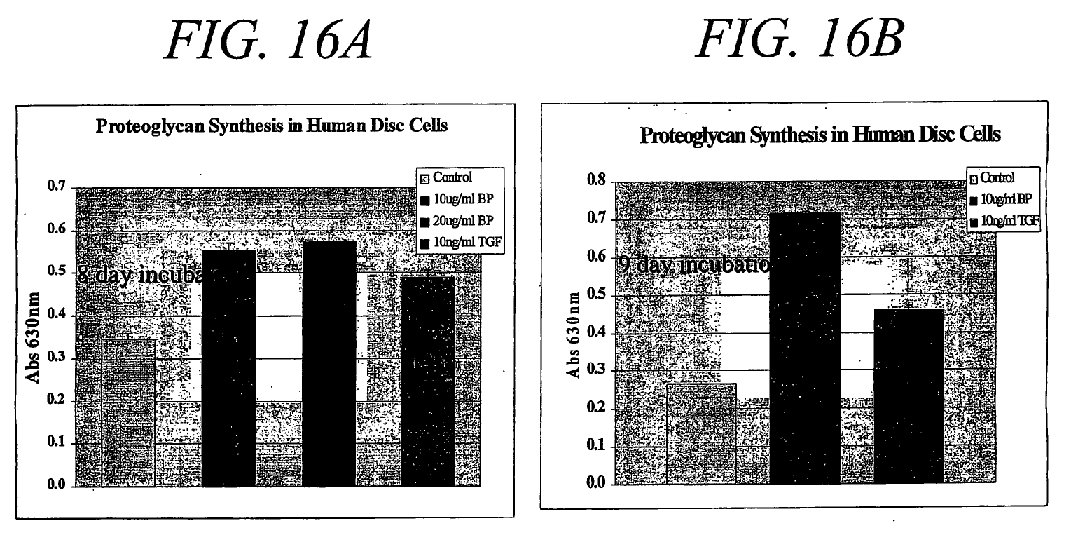

At 8 days (FIG. 16A), both growth factors exhibited a significant increase in Alcian blue staining over the control. TGF-β1 at 10 ng/ml had a 42% increase over the control. BP had a 60% increase at 10 μg/ml and 66% increase at 20 μg/ml over the control.

### 5.15 Example 15

### Effect of Exogenous Growth Factors on Proteoglycan Synthesis in Human Intervertebral Disc Nucleus Pulposus Cells

A second experiment to test the effects of TGF-β1 and BP on proteoglycan synthesis was performed on a different human patient from that described in Example 14. Human intervertebral disc cells obtained from another 40-year-old female patient were cultured as described in Examples 8 and 9 and seeded into 24 well plates. Growth factors were added after the cells were allowed to adhere overnight. TGF-β1 was tested at a concentration of 10 ng/ml; BP was tested at 10 μg/ml. After 6 and 9 days the cells were fixed and the amount of proteoglycans synthesized was measured by the Alcian blue assay as described in Example 10.

At 6 days cells stimulated with 10 ng/ml TGF-β1 produced 54% more proteoglycans than control, and 10 μg/ml BP increased production by 104% over the control. At 9 days (FIG. 16B), 10 ng/ml TGF-β1 increased production by 74% over controls, and 10 μg/ml BP increased production by 171% over the control.

### 5.16 Example 16

### Effect of Exogenous Growth Factors on Proteoglycan Synthesis in Baboon Intervertebral Disc Nucleus Pulposus Cells

TGF-β1 and BP were tested for their effects on stimulation of proteoglycan synthesis in baboon nucleus cells. Baboon intervertebral disc nucleus pulposus cells were obtained from a 7 year old male baboon, cultured as described in Examples 8 and 9, and seeded into a 24 well plate. The cells were allowed to adhere to the well surface before the addition of growth factors. The concentrations of growth factors tested were 10 μg/ml BP and 10 ng/ml TGF-β1. The dilutions were prepared in TCM. The cells were fixed after 4 and 8 days of continuous exposure to growth factors, and proteoglycan synthesis was detected by the Alcian blue assay as described in Example 10.

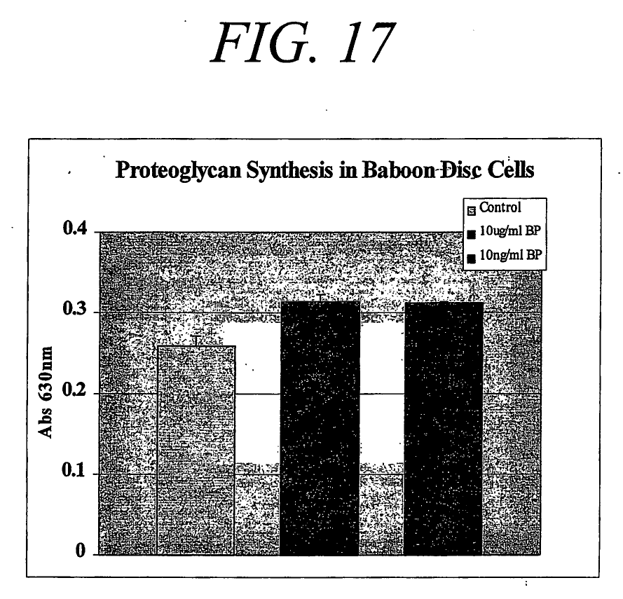

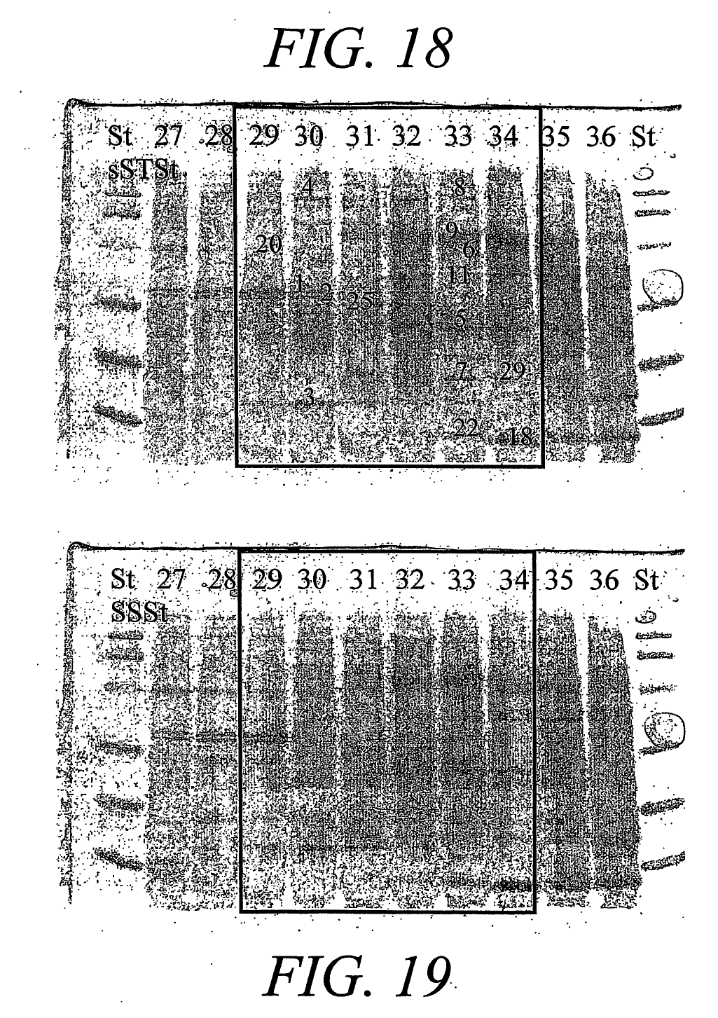

At 4 days there was no significant increase in proteoglycan synthesis between the different growth factors and the control. At 8 days (FIG. 17), TGF-β1 and BP significantly increased proteoglycan synthesis over the control, but the increase was only marginal. In particular, TGF-β1 produced a 21% increase over the control while BP produced a 22% increase over the control.

### 5.17 Example 17

### Staining of Seeded Matrix Material with Phalloidin

Cross-linked matrix seeded with living cells was stained with phalloidin to indicate the growth and proliferation of living cells into the matrix. The media was rinsed from the matrix with 3 PBS washes of 5 min each. The matrix was fixed for 1 hour at room temperature with 4% paraformaldehyde. The 4% paraformaldehyde was washed off with 3 PBS rinses. The matrix was treated with 0.1% Triton-X 100™ for 3 min and then washed with 3 PBS rinses. The matrix was then stained with phalloidin-conjugated rhodamine, made up in PBS, for 45 min. Excess phalloidin was washed off with PBS. The matrix was mounted on slides and viewed under fluorescence with filter of the range 530-550 nm.

### 5.18 Example 18

### Growth and Proliferation of Sheep Intervertebral Disc Nucleus Pulposus Cells into Non—Homogenized Matrix with BP Growth Factor

Ingrowth and proliferation of growth factor stimulated sheep intervertebral disc nucleus pulposus cells into the matrix of the present invention was investigated. Cross-linked matrix material obtained prior to the lyophilization step of Example 1 was cut into square pieces 75 mm on each side and sterilized in 70% ethanol for 3 hours. Remaining steps in the protocol were performed under aseptic conditions.

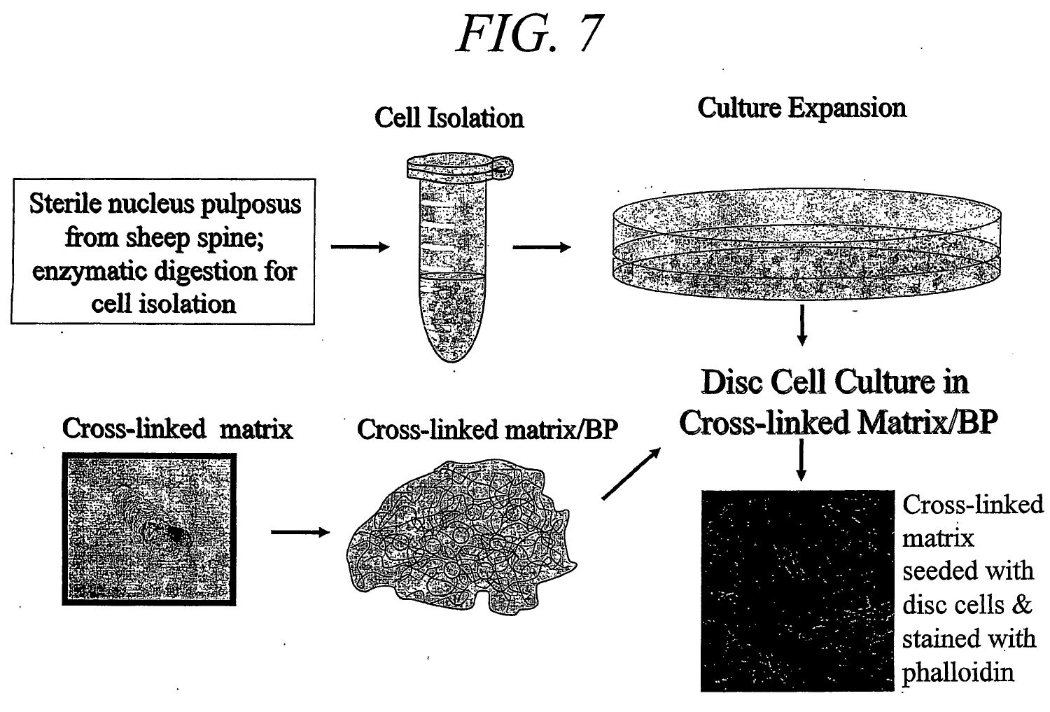

Ethanol was removed from the matrix with two 1-hour washes in sterile PBS, followed by a one hour wash in TCM. The matrix pieces were then suspended overnight in TCM having BP concentrations of 20 ng/ml and 20 μg/ml. The control was cross-linked matrix suspended in 20 μg/ml BSA (bovine serum albumin). Each matrix piece was then placed in a well of a 24-well plate and seeded with TCM containing sheep intervertebral disc nucleus cells at 40,000 cells/ml. The cells were allowed to grow into the matrix and the TCM was changed every 2-3 days. Sample matrix pieces were fixed at 3, 6 and 9 days and stained with phalloidin as described in Example 17. The process is illustrated in FIG. 7.

Infiltration of sheep nucleus pulposus cells into the matrix was observed at all of the 3, 6 and 9 day time points, indicating that the matrix was biocompatible. The number of cells observed per field was higher at 6 and 9 days, indicating that the cells were proliferating into the matrix. More cells were observed in matrix pieces that had been suspended in TCM containing BP than in controls having no growth factor. BP at 20 μg/ml produced the greatest infiltration and proliferation of cells into the matrix.

### 5.19 Example 19

### Growth and Proliferation of Sheep Intervertebral Disc Nucleus Pulposus Cells into Homogenized Matrix with BP Growth Factor

A further investigation of the ingrowth and proliferation of growth factor stimulated sheep intervertebral disc nucleus pulposus cells into the matrix of the present invention was made using homogenized matrix, as opposed to the non-homogenized matrix in Example 18. Cross-linked matrix material obtained prior to the lyophilization step of Example 1 was homogenized using a tissue homogenizer, and sterilized in 70% ethanol for 3 hours. All subsequent steps in the protocol were under aseptic conditions.

The homogenized matrix was centrifuged at 3200 rpm for 10 min and the supernatant was discarded. The pelleted matrix was rinsed with two 1-hour PBS washes, followed by a 1-hour TCM wash. Between each wash the matrix was centrifuged, and the supernatant was discarded. The pelleted matrix was then suspended overnight in TCM having BP concentrations of 20 ng/ml and 20 μl/ml. The control was cross-linked matrix suspended in 20 μg/ml BSA.

The TCM/matrix mixture was then centrifuged and the supernatant was discarded. The matrix pellet was suspended in TCM containing sheep intervertebral disc nucleus cells, obtained according to the procedure in Examples 8 and 9. The matrix/cell suspension was pipetted into wells of a 24-well plate. The TCM was changed every 2-3 days. The homogenized matrix seeded with cells was fixed at 4 days and stained with phalloidin as described in Example 17. The process is illustrated in FIG. 7.

After 4 days, the layer of cross-linked matrix soaked in 20 μg/ml BP and seeded with cells had contracted to form a rounded clump of compact tissue. This tissue was comprised of both the original cross-linked matrix and the newly synthesized matrix produced by the infiltrated cells. There were very few cells adherent to the well surface, indicating that most cells had infiltrated the matrix. This conclusion was reinforced by the dense infiltration of cells into the matrix as visualized by phalloidin staining. The cells had assumed a rounded morphology which is characteristic of nucleus chondrocytic cells, indicating reversion to their original morphology. Cells had also grown into matrix soaked in 20 ng/ml BP by 4 days, but cell ingrowth was not as dense as in the matrix soaked in 20 μg/ml BP.

The control matrix suspended in BSA also had cells infiltrating into it, but it was the least populated among the different dilutions.

### 5.20 Example 20

### In Vivo Evaluation of Cross-Linked Matrix and Bone Protein (BP) Growth Factor for Nucleus Pulposus Regeneration in an Ovine Lumbar Spine Model

Pilot studies were conducted to evaluate preparative and surgical methods for the implantation of the cross-linked matrix containing BP growth factors into the intervertebral disc space of the sheep lumbar spine, to evaluate whether implantation of the matrix with growth factors arrests degeneration and/or stimulates regeneration of nucleus pulposus in a sheep disc degeneration model over a period of six months, and to assess the antibody- and cell-mediated immune response in sheep to the matrix/BP combination.

5.20.1 Study 1

One-half gram (0.5 g) of cross-linked, lyophilized and pulverized matrix prepared as described in Example 1 was rehydrated and sterilized by two 4 hour rinses in 70% isopropanol. The matrix was centrifuged and pelleted, and then rinsed in sterile PBS three times for 2 hr each to remove the isopropanol. The rehydrated matrix was again centrifuged and pelleted.

Bone Protein (BP) prepared according to U.S. Pat. Nos. 5,290,763 and 5,371,191 was obtained from Sulzer Biologics, Inc. (Wheat Ridge, Colo.) in a lyophilized form. Two milligrams (2 mg) of BP was suspended in 100 μl dilute 0.01 M hydrochloric acid to produce a 20 mg/ml BP stock solution. The BP stock solution was diluted to 100 μg/ml in sheep serum and the BP/serum suspension was sterile-filtered through a 0.2 micron filter. Next, 1.0 ml of the sterile BP/serum suspension was added to 1.0 ml of the rehydrated matrix described above to obtain a final concentration of 50 μg BP per ml of cross-linked, rehydrated matrix/serum suspension. At the time of surgery, one aliquot (0.5 ml) of the rehydrated matrix/BP/serum suspension was loaded into a sterile 3-ml pressure control syringe with an 18 or 20 gauge needle for injection.

Three sheep were anesthetized and the dorsolateral lumbar area prepared for surgery. Blood was drawn from each sheep pre-operatively, centrifuged, and serum collected for immunology studies. A ventrolateral, retroperitoneal approach was made through the oblique abdominal muscles to the plane ventral to the transverse processes of the lumbar spine. The annuli fibrosi of intervertebral discs L3-4, L4-5, and L5-6 were located, soft tissues retracted, and a discrete 5 mm deep by 5 mm long incision was made into both L3-4 and L5-6 discs. The intervening, middle L4-5 disc remained intact to serve as an intra-operative control. Following annulus stab procedures, the musculature and subcutaneous tissues were closed with absorbable suture. After postoperative recovery, sheep were allowed free range in the pasture.

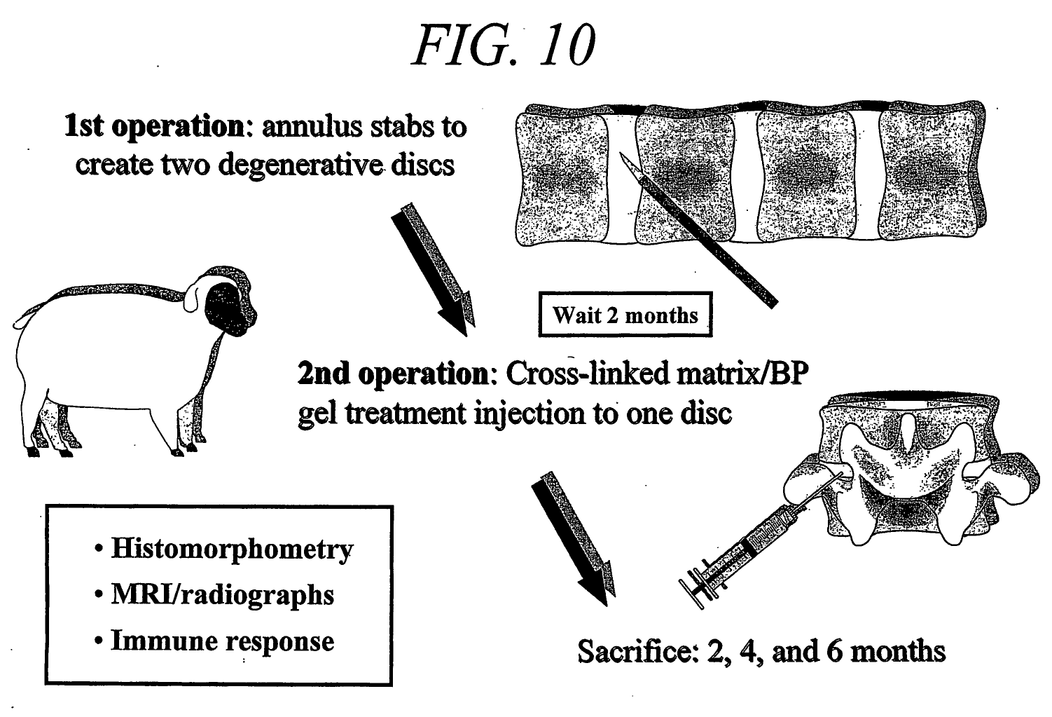

Two months after the annulus stab surgical procedures, the sheep were operated upon a second time. After anesthesia and preparation for surgery, the three operated lumbar spine levels were again exposed. Two-hundred microliters (200 μl) of the prepared test material (i.e., rehydrated matrix/BP/serum suspension) was injected into the intradiscal space of one (L5-6) of the experimentally-damaged discs. The second operated disc (L3-4) served as a sham-treated degenerative disc; the syringe needle punctured the annulus but no material was injected. After disc treatments, the musculature and subcutaneous tissues were closed with absorbable suture. Following postoperative recovery, sheep were allowed free range of movement. The study design is diagrammatically represented in FIG. 10.

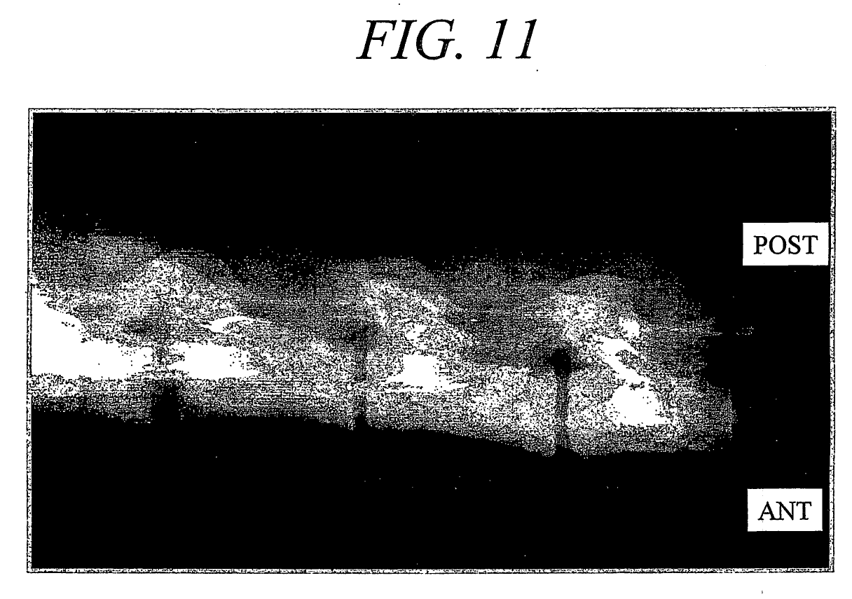

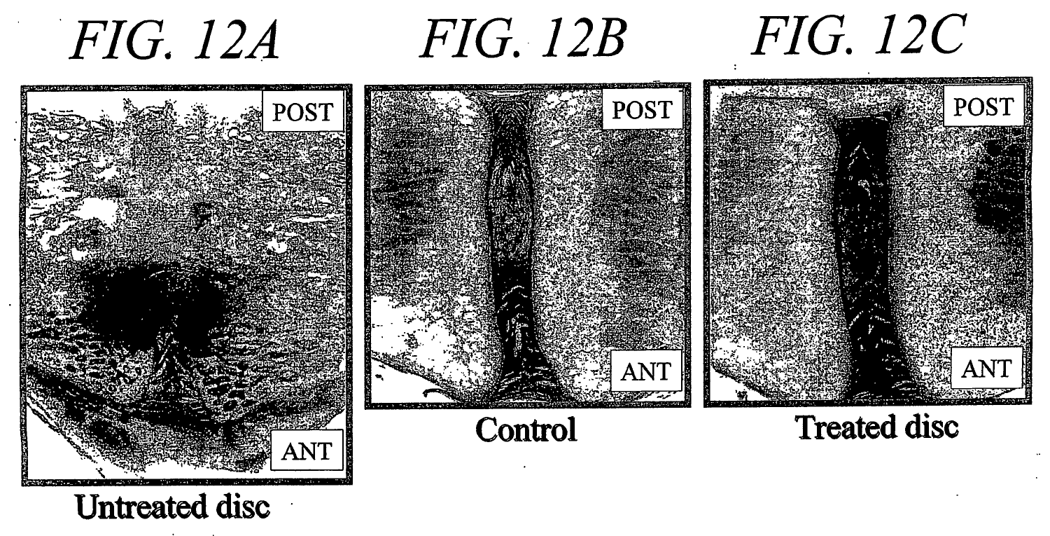

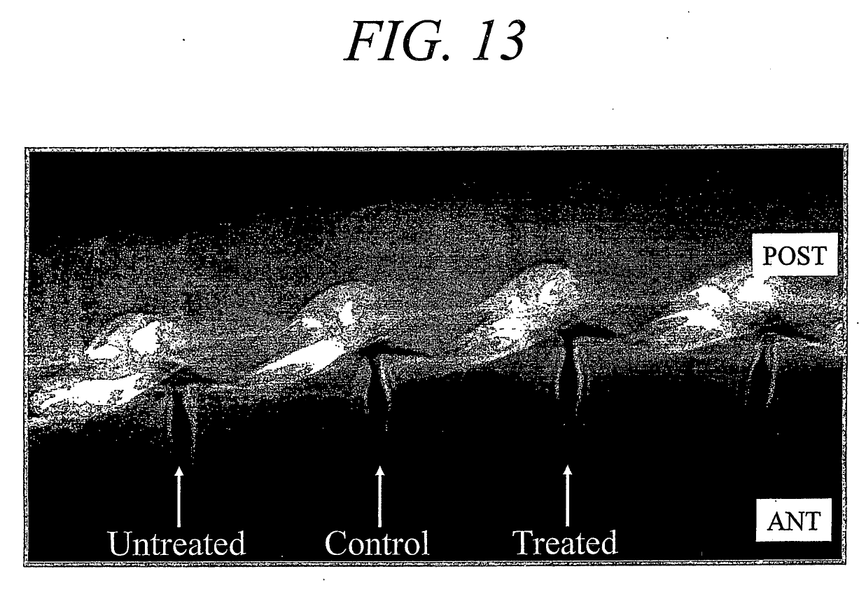

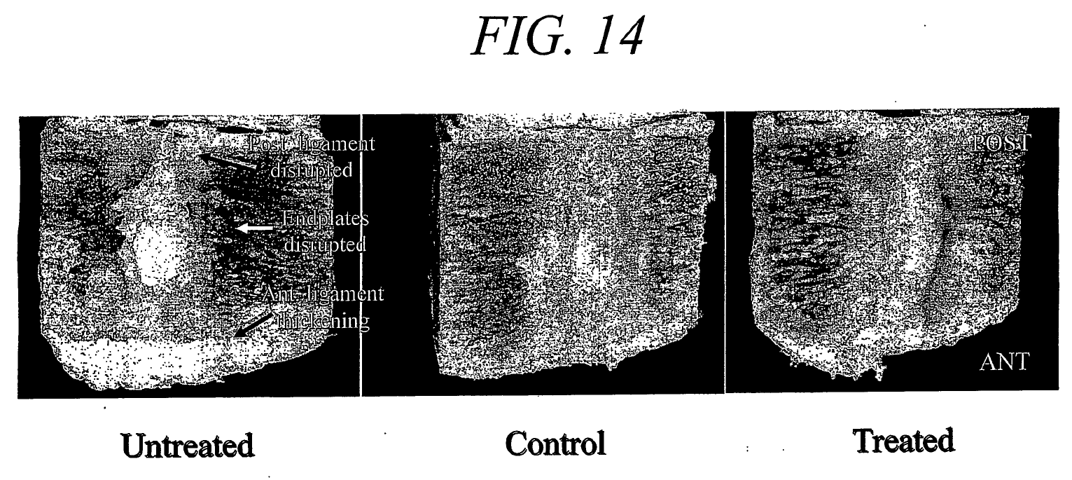

The sheep were sacrificed at 2, 4, and 6 months after the second surgery. The radiograph from the 2 month sheep showed a degenerative appearance of the untreated disc but a normal appearance in the control and treated discs (FIG. 11). Histological analysis of the 2 month sheep as illustrated in FIG. 12A, FIG. 12B and FIG. 12C confirmed extensive degeneration within the sham-treated, stab-induced degenerative disc. In both the control disc and the matrix/BP-treated disc, a normal sized gelatinous nucleus and regular, compact annulus were observed. In the 4 month and 6 month sheep, no obvious changes were seen in the radiograph of the three discs. A radiograph of the 4 month sheep is shown in FIG. 13. However, on gross dissection in the 4 month sheep, the sham-treated disc exhibited obvious gross degeneration while the control and treated discs were normal in appearance (FIG. 14). In the 6 month sheep, there were no gross differences between the sham-treated, control, and treated discs.

Although there was some variation in the rate of degeneration using the annulus stab technique (i.e., the absence of clear degeneration in the 6 month sheep), these results suggest that the cross-linked matrix/BP treatment may protect against or impede the progress of stab-induced degeneration in sheep intervertebral discs.

5.20.2 Study 2

For a second study, matrix material was rehydrated and combined with BP and serum to produce a matrix/BP/serum suspension as described in Section 5.20.1 (Study 1).

Twelve sheep were anesthetized and the dorsolateral lumbar area prepared for surgery. Blood was drawn from each sheep pre-operatively, centrifuged, and serum collected for immunology studies. A ventrolateral, retroperitoneal approach was made through the oblique abdominal muscles to the plane ventral to the transverse processes of the lumbar spine. The annuli fibrosi of intervertebral discs L1-2, L2-3, L3-4, L4-5, and L5-6 were located, soft tissues retracted, and a small diameter hole punched through the annulus using a syringe needle in 4 of the 5 discs. A small curette was then placed through the hole into the intradiscal space to remove a discrete portion of nucleus pulposus from each of the four discs in each sheep. In 2 of the 4 damaged discs, 0.5 ml of the matrix/BP/serum suspension was injected into the intradiscal spaces and the needle punctures were sealed off with ligament sutured over them. The immediate injection of this suspension was considered an “acute” treatment protocol. The 2 other damaged discs were left untreated at that time but were sealed off with ligament sutured over the needle punctures. The intervening, middle L3-4 disc remained intact in all sheep spines to serve as an intra-operative control. Following these procedures, the musculature and subcutaneous tissues were closed with absorbable suture. After postoperative recovery, sheep were allowed free range.

Six weeks after the first surgery to remove portions of the nucleus pulposus, the sheep were operated upon a second time. After anesthesia and preparation for surgery, the five operated lumbar spine levels were again exposed. In one of the two remaining nontreated discs which had been damaged six weeks before, 0.5 milliliters of the prepared test material (i.e., rehydrated matrix/BP/serum suspension) was injected into the intradiscal space of the disc. The injection of this suspension six weeks later into a damaged disc was considered a “delayed” treatment protocol. The second nontreated damaged disc served as a sham-treated degenerative disc; the syringe needle punctured the annulus but no material was injected. The treatment method used in each of the four experimentally-damaged discs was randomized for location within the spines. That is, except for the intact control disc (L34), the locations of an “acute” treatment disc, a “delayed” treatment disc, or a nontreated, damaged disc, were randomly assigned to one of the four different lumbar disc levels. After disc treatments, the musculature and subcutaneous tissues were closed with absorbable suture. Following postoperative recovery, sheep were allowed free range.

The sheep were sacrificed at 2, 4, and 6 months after matrix/BP/serum injections and the spines were fixed for histology in formalin. Cross-sections were taken from plastic-embedded discs, stained with H & E and Saffranin-O, and evaluated for chondrocyte proliferation (cloning), proteoglycan staining intensity, level of fibrosis, and level of ossification. An evaluation of the “acute” treatment discs, “delayed” treatment discs, sham-treated, and control discs was made in a blinded fashion and ranked +1, +2, or +3 (low, medium, or high) for each parameter listed above. Semiquantitative evaluation of the histological results was compared in 2 month, 4 month, and 6 month sheep for both the “acute” and “delayed” (6 week) treatments.

The results demonstrated overall that injected matrix +BP stimulated chondrocyte cloning and accumulation of Saffranin-O staining of glycosaminoglycans in the nucleus matrix of damaged discs. In particular, the extent of regenerative repair was much greater in both “acute” treatment discs and “delayed” treatment discs, compared to that observed in non-treated, damaged discs. This greater level of repair in matrix/BP-treated discs was statistically significant at the 0.01 level of confidence. There was also less fibrosis and ossification seen in the acute and delayed treatment discs compared to the non-treated discs.

A significant difference was also noted between the “delayed” treatment discs and the “acute” treatment discs in the level of proteoglycan staining. For example, Saffranin-O staining as an index to proteoglycan synthesis and content in the nucleus matrix was greater in the “delayed” matrix/BP-treatment discs than in the “acute” matrix/BP-treatment discs. Additional benefits apparent in the histological evaluation, which were associated with “delayed” treatment with matrix/BP, were an overall lack of bony transformation (ossification) or fibrous tissue accumulation (fibrosis) within the treated discs compared to the non-treated, damaged discs. In general, the results in Study #2 support and elaborate earlier indications from Study #1 that treatment of damaged discs with the cross-linked matrix/BP may protect against or impede the progress of degeneration in experimentally-damaged intervertebral discs.

### 5.21 Example 21

### Synthesis of Photo-Polymerizable Hyaluronic Acid; Derivative Modification of Hyaluronic Acid with Glycidyl Methacrylate

Fluid matrices for intervertebral disc treatment comprising decellularized, crosslinked nucleus pulposus material obtained from a porcine donor animal, combined with an in situ cross-linkable polymeric viscosity control agent have been synthesized. Nucleus pulposus tissues from other vertebrates (i.e., human, bovine, ovine, etc.) may also be used. The viscosity control agent comprises hyaluronic acid functionalized with a cross-linkable moiety. Other cross-linkable proteoglycans may be used as alternative viscosity control agents. Non-proteoglycan polymers that are crosslinkable (e.g., functionalized polyalkylene glycols) may also be used as viscosity control agents, either alone or in combination with crosslinkable proteoglycans, but are not preferred. Proteoglycan viscosity control agents are preferred because they can also function as cell enhancement agents. The resulting matrices provide biocompatible hydrogels useful in augmenting the nucleus pulposus space in a degenerated disc, and which also provide a potential therapeutic substance to regenerate the nucleus pulposus of a patient to whom the matrices are delivered.

Human recombinant Hyaluronic Acid (“rhHA”) was purchased from Genzyme Biosurgery, Inc. Hyaluronic acid derived from animal tissue sources was purchased from Sigma-Aldrich. For convenience, hyaluronic acid of either human or animal origin as “HA.” Glycidyl methacrylate (GM), triethyl amine, acetone, tetra butyl ammonium bromide, vinyl caprolactam (VC), and vinyl pyrrolidinone (VP) were purchased from Sigma-Aldrich. Irgacure 2959 was obtained from Ciba-Geigy. All other chemicals and equipment were of reagent grade, were used as received, and were obtained from standard suppliers including Fisher Scientific, VWR Scientific, and Sigma-Aldrich.

In a 500-ml Erlenmeyer flask, 1 g sodium hyaluronate (“HA”) was dissolved in 100 ml deionized water at room temperature to make a 1% (wt./vol.) HA solution. Unless otherwise stated, percentage concentrations are wt./vol. Two (2) ml of liquid glycidyl methacrylate, 2 ml of liquid triethylamine, and 2 grams of tetra butyl ammonium bromide were slowly added to the 1% HA solution while stirring at room temperature for 30 min. The glycidyl methacrylate-modified HA (GM-HAM) reaction mixture was then allowed to stand at room temperature for 24 hrs without agitation. Finally, the flask containing GM-HAM solution was heated to between 50° C. and 60° C. in a water bath for 1 hour.

After the GM-HAM reaction mixture was allowed to cool to room temperature, the modified HA was precipitated out of solution by addition of 1.5 L acetone. The GM-HAM precipitate was rinsed 2-3 times in fresh acetone, the acetone evaporated off, and the GM-HAM re-dissolved in 100 ml deionized water. This final GM-HAM solution was frozen at −80° C. and lyophilized to powder. The lyophilized GM-HAM was stored at 4° C. until use.

The addition of acrylic bonds to the hyaluronic acid backbone in the foregoing reaction renders the HA susceptible to free radical polymerization or, more preferably, co-polymerization and subsequent cross-linking of photo-polymerizable hyaluronic acid.

### 5.22 Example 22

### Photo-Polymerization of Gm-Ham Under Varying Conditions

Solutions of 1-3% (wt./vol.) GM-HAM were made in phosphate buffered saline containing between 500 ppm to 2000 ppm of photo-initiator (Irgacure 2959) and between 1 μl/ml to 10 μl/ml of the co-monomer vinyl pyrrolidinone (“VP”). The VP was included as co-monomer to reduce the time necessary to cross-link the GM-HAM and thereby increase the viscosity of the combined matrix with nucleus pulposus tissue. Without being bound by any particular theory, it is believed that the VP molecules facilitate cross-linking of the large GM-HAM molecules by providing a bridging moiety that obviates steric hindrances between the reactive sites of the large GM-HAM molecules.

Samples (70-100 μl) of the GM-HAM/VP/initiator mixture were exposed to long wave ultraviolet light (Black-Ray lamp, UV wavelength—365 nm) for varying periods of time to determine optimum parameters necessary to fully crosslink these hydrogels by a polymerization-type crosslinking reaction. Table 1 lists representative concentrations of substrate GM-HAM, co-monomer, and initiator that were sufficient in combination to produce hydrogels within a nominal time constraint of 2 min of UV light exposure.

Relatively short polymerization times are required to minimize trauma to the patient. Polymerization times of less than 300 seconds are preferred, with times of 1-100 seconds considered to be optimum.

TABLE 1
POLYMERIZATION CONDITIONS AND FORMULATION
INGREDIENTS FOR PHOTO-POLYMERIZABLE HYDROGELS

| GM-HAM | | | Time | |
|---|---|---|---|---|
| (% wt./vol.) | Irgacure 2959 | VP (μl/ml) | (min:sec) | Description |
| 2.5 | 1000 ppm | 2 | 1:30 | Firm gel |
| 2.5 | 1000 ppm | 4 | 1:20 | Firm gel |
| 2.5 | 1000 ppm | 6 | 2:00 | Firm gel |
| 2.5 | 2000 ppm | 4 | 1:05 | Firm gel |
| 2.0 | 2000 ppm | 8 | 1:15 | Firm gel |

### 5.23 Example 23

### Encapsulation of Living Sheep Nucleus Chondrocytes in Combination Hydrogels of GM-HAM and Photo-Oxidized PNP Matrix (PF)

A 3% (wt./vol.) solution of GM-HAM was prepared in PBS containing 2000 ppm Irgacure 2959 and 6 μl/ml vinyl caprolactam. Lyophilized photo-crosslinked nucleus pulposus (“PNP”) matrix produced substantially in accordance with Example 1, supra, was rehydrated in PBS. The matrix was then sterilized by centrifugation and hydration in 70% (vol./vol.) ethanol. It was then re-centrifuged to remove the ethanol, and rinsed in PBS to remove the ethanol and rehydrate solely in PBS. One hundred (100) mg of rehydrated PNP matrix occupied a volume of 1 ml thereby giving a 10% wt./vol. solution. The 3% GM-HAM solution was diluted 1:1 with either: (1) sterile decellularized 10% PNP; (2) sterile, decellularized, 5% PNP (obtained by adding equal amounts of PBS to 10% PNP); or, (3) sterile PBS to give final solutions of: (1) 1.5% GM-HAM/5% PNP (with initiator and vinyl caprolactam); (2) 1.5% GM-HAM/2.5% PNP (with initiator and vinyl caprolactam); and (3) 1.5% GM-HAM/PBS (also with initiator and vinyl caprolactam), respectively.

Sheep nucleus chondrocytes (SNCs) were suspended in the GM-HAM/PBS and GM-HAM/PNP matrix solutions at a density of 2×10⁶ cells/ml. The SNC/matrix suspension was poured into molds and photo-polymerized using long-wave ultraviolet light at 365-nm wavelength. After 2 min UV exposures, the SNCs were encapsulated in the polymerized hydrogels. The hydrogels were then placed in Transwell® inserts (Corning, Inc.) which were placed in separate wells of a 12-well plate and incubated in growth media (DMEM/F-12 supplemented with 10% FBS, 50 μg/ml ascorbic acid and antibiotics).

Hydrogels from the three groups were removed from the growth media at various time points (0, 1, 2, 6, 14, and 21 days) and stained with a commercially available Live/Dead fluorescent stain to assess viability of SNCs (live cells appeared green, dead cells appeared red). Microphotographs of the cells were taken at each time point listed above. Polymerization times to form firm hydrogels were as follows: 1.5% GM-HAM alone polymerized in 50 sec; 1.5% GM-HAM+2.5% PNP material polymerized in 1 min; 1.5% GM-HAM+5.0% PNP material polymerized in 1 min, 20 sec.

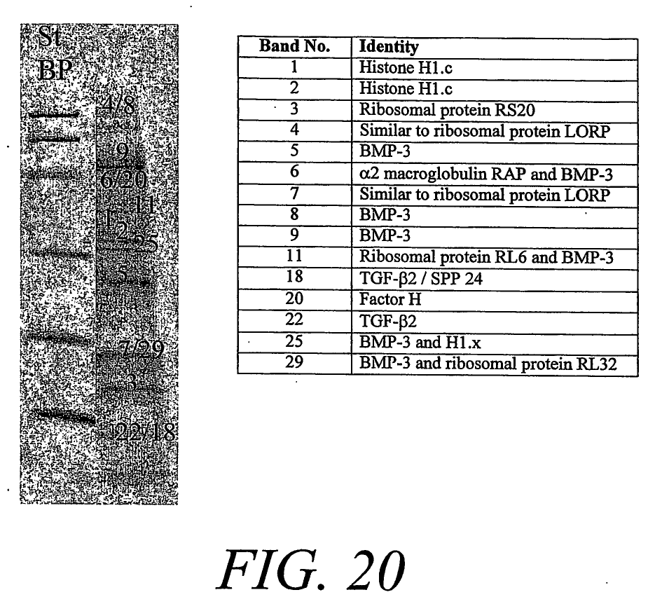

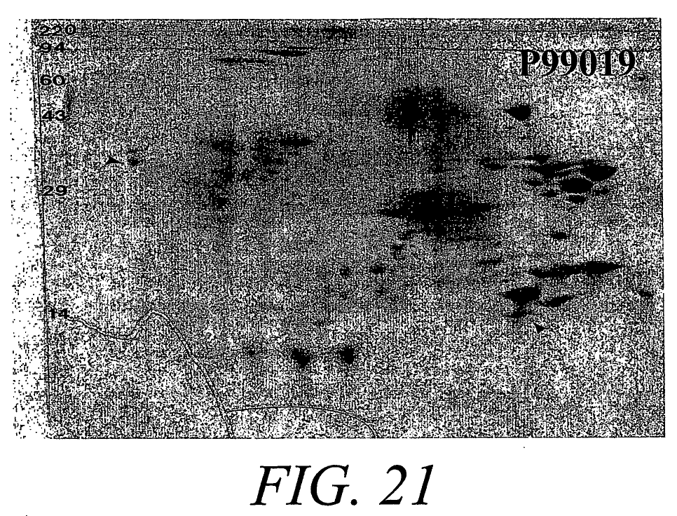

Data for immediate (0 hr) incubation in the three hydrogel formulations showed excellent initial SNC viability. Survival of SNCs dropped to 50% by 24 hours in GM-HAM gels, but remained very high in gels which included PF matrix materials. Table 2 list the viability of cells after polymerization and encapsulation in the crosslinked matrix. The high viability of cells immediately and after 24 hours indicates the cytocompatibility of the GM-HAM/PNP matrix formulations. FIG. 20 and FIG. 21 provide photomicrographs that demonstrate the enhanced survivability of SNCs in the PNP matrix over crosslinked GM-HAM alone.

TABLE 2
SNC VIABILITY IN FORMED HYDROGELS

| Datapoint | Matrix | % Viability |
|---|---|---|
| 0 hr | 1.5% GM-HAM | 82.42 |
| 0 hr | 1.5% GM-HAM/25% PF | 87.10 |
| 0 hr | 1.5% GM-HAM/50% PF | 90.75 |
| 24 hr | 1.5% GM-HAM | 50.44 |
| 24 hr | 1.5% GM-HAM/25% PF | 85.90 |
| 24 hr | 1.5% GM-HAM/50% PF | 97.08 |

It is understood that preferred compositions described in the disclosure can be polymerized in situ inside the disc space using minimally invasive surgical devices. The light can be transmitted into the disc space using fiber-optic UV light systems similar to commercially available instruments developed for dental applications and surgical adhesive applications such as FocalSeal™ (Genzyme Biosurgery Inc., Cambridge Mass.).

Modified hyaluronic acid is particularly preferred as a viscosity control agent since it is a major component of disc nucleus materials. However, the GA-HAM materials used in the examples as a viscosity control agent and cell enhancement agent may be substituted with other water soluble macromonomers such as polyethylene glycol based macromonomers. Moreover, although Irgacure 2929 was used in this preferred embodiment, free radicals to initiate the cross-linking co-polymerization reaction can be generated by other free radical initiators or photo-initiators known in the art of polymer chemistry, e.g., eosin/triethanolamine. Water soluble thermal initiators such as ammonium persulfate can also be used. Irgacure is preferred because it is soluble (up to 3%) in water and aqueous buffers such as PBS, and it requires very small amounts to initiate crosslinking. Finally, although VP was used as the co-monomer in Example 22, other co-monomers such as vinyl caprolactam, acrylic acid, polyethylene glycol acrylates, and methacrylate esters may also be used. Water soluble co-monomers are preferred. For other macromers and initiators see, e.g., Pathak et al. (1993). Chemical modification of native HA in nucleus pulposus is also possible using the same chemistry as described in Example 1. Exogenous materials are preferred, however.

In preferred embodiments, modified or unmodified nucleus pulposus material derived from animal or human sources is combined by, e.g., mixing, with a solution that can be polymerized in situ inside the intervertebral disc space (or other space inside the body). Example 21 describes the synthesis of photopolymerizable hyaluronic acid.

Matrix compositions useful in treating intervertebral disc impairment in vertebrates, including humans, may be prepared according to the foregoing descriptions and examples. While various embodiments of the inventions have been described in detail, modifications and adaptations of those embodiments will be apparent to those of skill in the art in view of the present disclosure. However, such modifications and adaptations are within the spirit and scope of the present inventions, as set forth in the following claims.

### 5.24 Example 24

### Encapsulation of Living Human Articular Chondrocytes in Combination Hydrogels of GM-HAM and Photo-Oxidized PNP Matrix (PF) and Alginate Beads

1.5% GM-HAM/PBS and 1.5% GM-HAM/2.5% PNP were prepared as described in Example 23. Normal human articular chondrocytes (NHACs) purchased from Cambrex Bio Science Walkersville, Inc. were suspended in the matrix formulations at a cell density of 2×10⁶ cells/ml. The NHAC/matrix suspension was poured into molds and photo-polymerized using long wave ultraviolet light at wavelength of 365 nm. After 1 minute of UV exposure, NHACs were encapsulated in the hydrogels. The hydrogels were then placed in Transwell inserts (Corning, Inc.) which were placed in separate wells of a 12-well plate and incubated with chondrocyte differentiation media (Cambrex Bio Science). NHACs were also suspended in 1.2% alginate beads as a positive control. Alginate beads were formed by polymerization of NHAC/alginate suspension, expressed through an 18-gauge needle, in CaCl₂ solution.

Samples from the three groups were removed from the growth media at various time points (0, 1, 7, 14, and 28 days) and stained with a commercially available Live/Dead fluorescent stain to assess viability of SNCs (live cells appeared green, dead cells appeared red). Microphotographs of the cells were taken at each time point listed above.

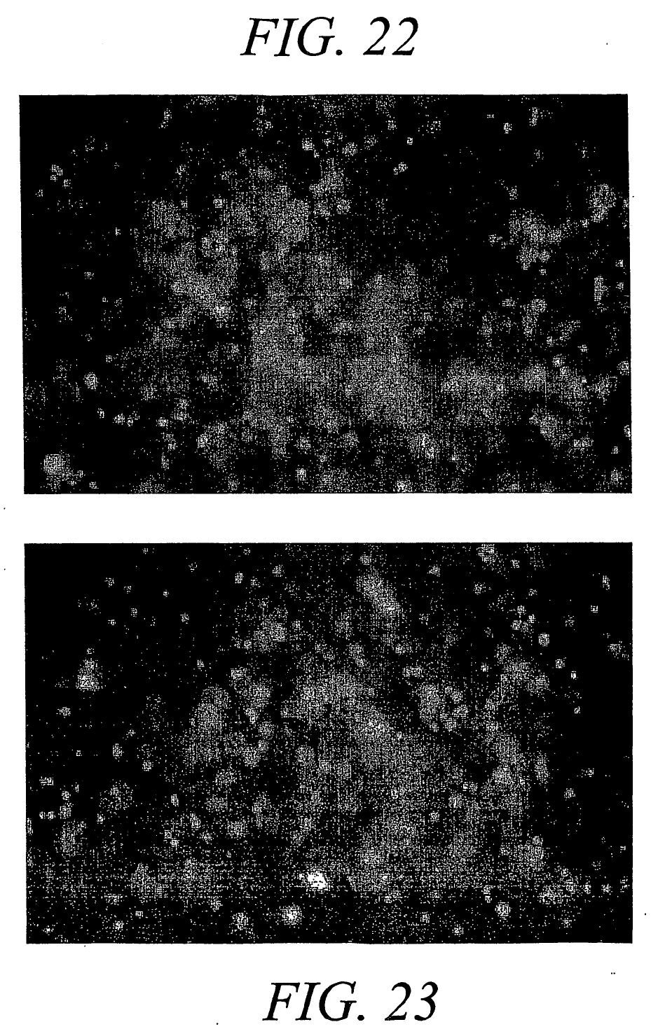

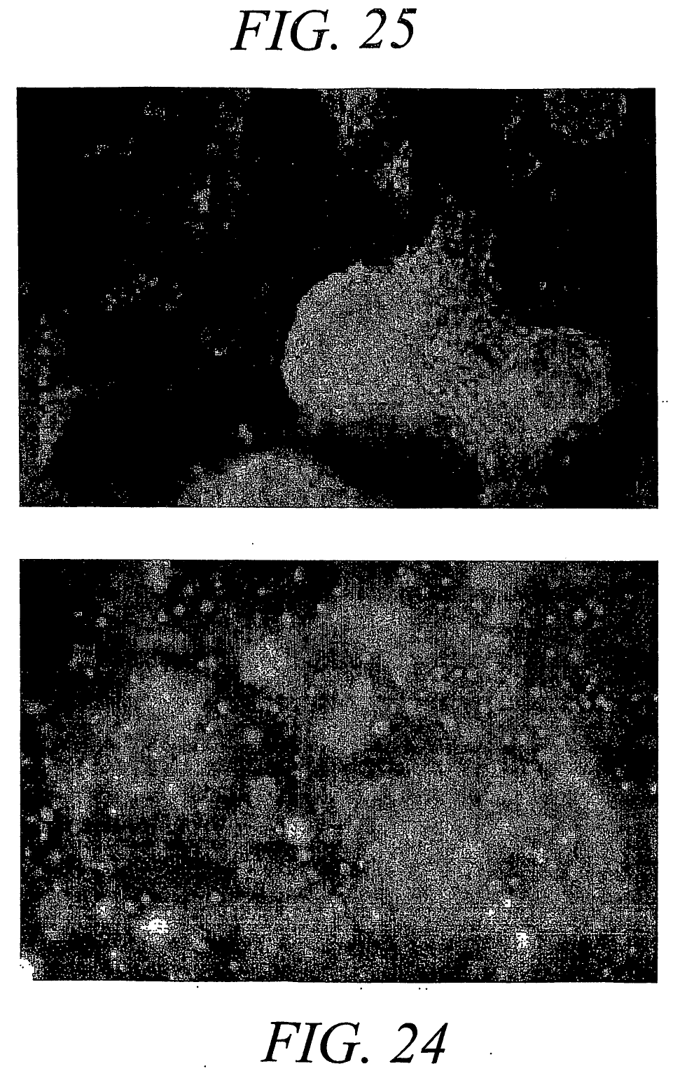

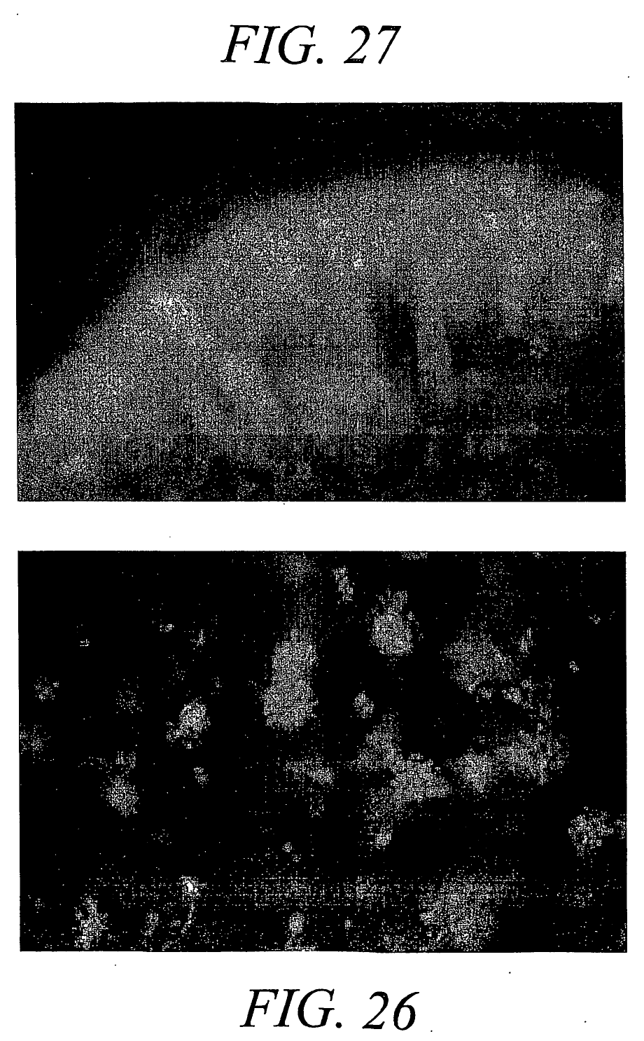

Data for immediate (0 hr) incubation in the three formulations showed excellent initial NHAC viability as shown in FIG. 22, FIG. 23 and FIG. 24. Survival remained very high in all the formulations after 24 hours. The high viability of cells immediately and after 24 hours indicates the cytocompatibility of the GM-HAM and GM-HAM PNP matrix formulations. FIG. 25, FIG. 26 and FIG. 27 provide photomicrographs which demonstrate that NHACs remained viable and continued to proliferate and migrate across both the hydrogels as in the alginate beads through 28 days of culture.

### 5.25 Example 25

### Cytotoxicity of Residual Photo Polymerization Components Released from Combination Hydrogels of GM-HAM and Photo-Oxidized PNP Matrix (PF)

Sheep nucleus cells (SNCs) were seeded in 24-well plates at 25,000 cells/well and incubated with Tissue Culture Medium (hereinafter referred to as “TCM”) comprising DMEM/F-12 culture medium supplemented with 10% heat inactivated fetal bovine serum, 0.25% penicillin, 0.4% streptomycin, 0.001% amphotericin B, and 50 μg/ml ascorbic acid for 24 hours. 1.5% GM-HAM and 1.5% GM-HAM/2.5% PNP monomer solutions were prepared as mentioned in Example 23. The monomer solution was poured into molds and photo-polymerized using long-wave ultraviolet light at 365 nm wavelength. Polymerized hydrogels were placed into tissue culture inserts which were then placed above seeded SNCs. Cytotoxicity of unpolymerized matrix was also determined, by including inserts containing unpolymerized monomer solution. SNC seeded wells containing empty inserts served as controls. SNCs and inserts were completely immersed in TCM. After a 24 hour exposure, inserts were removed and viability of cells was assessed with the MTS (3-(4,5-dimethylthiazol-2-yl)-5-(3-carboxymethoxyphenyl)-2-(4-sulfonphenyl)-2H-tetrazolium) Assay. The MTS assay measures viability of cells by a calorimetric method, wherein the MTS tetrazolium compound is bio reduced by cells into a colored formazan product which is soluble in TCM. The conversion is accomplished by dehydrogenase enzymes present in metabolically active cells.

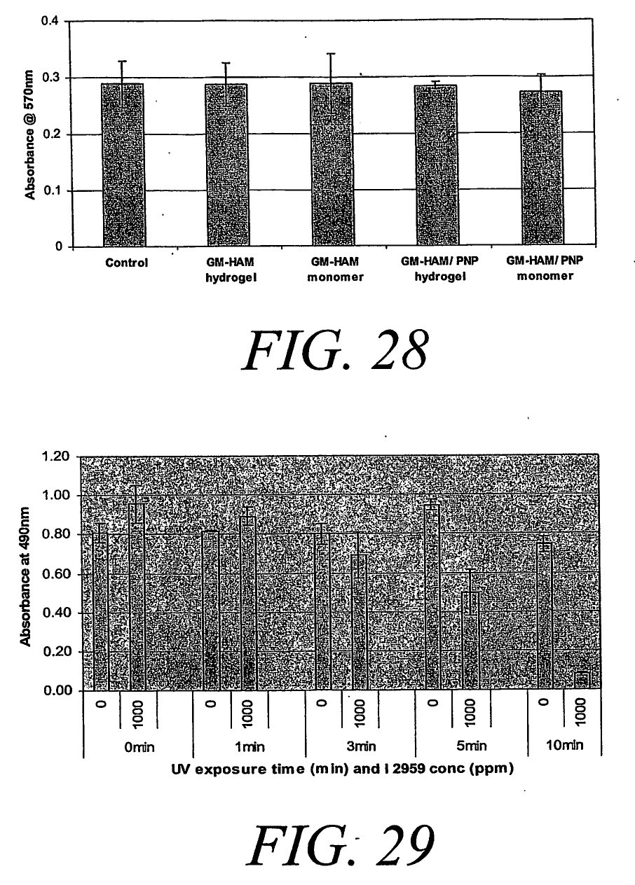

The graph in FIG. 28 depicts the absorbance of TCM at 570 nm for the various test materials. Absorbance at 570 nm directly correlates to the amount of formazan product produced by viable cells. From these results it can be concluded that neither the polymerized hydrogels nor unpolymerized monomer solutions were cytotoxic. Therefore, the photo initiating system constituents (Irgacure 2959, vinyl caprolactam) in the monomer solution and polymerized hydrogels as well as the residual free radicals released from the polymerized hydrogels are cytocompatible.

### 5.26 Example 26

### Cytotoxicity of the Photo-Initiating System on Living Sheep Nucleus Chondrocytes

Sheep nucleus chondrocytes were seeded in a 12-well plate at 20,000 cells/well and incubated with TCM. After allowing cells to adhere for over 48 hours, spent media was replaced with TCM containing 0 or 1000 ppm Irgacure 2959. SNCs were then exposed to long wave ultraviolet light at 365 nm wavelength for different lengths of time, followed by incubation at 37° C. MTT assay was performed at 1, 5, and 24 hours post UV exposure to assess viability of cells. The principle of the assay is similar to the MTS assay mentioned before, except that a different tetrazolium compound is used in this method.

As observed in FIG. 29, UV light and Irgacure 2959 by themselves were not significantly cytotoxic. Production of free radicals by the interaction of UV light with Irgacure 2959 did cause cell death, but it was significant only at 5 and 10 min of UV exposure. The current photo polymerization times of GM-HAM and GM-HAM/PNP matrix are under 3 min of UV exposure, which are not cytotoxic.

### 6. REFERENCES

The following references, to the extent that they provide exemplary procedural or other details supplementary to those set forth herein, are specifically incorporated herein by reference.
U.S. Pat. No. 4,772,287
U.S. Pat. No. 4,904,260
U.S. Pat. No. 5,047,055
U.S. Pat. No. 5,147,514
U.S. Pat. No. 5,171,280
U.S. Pat. No. 5,171,281
U.S. Pat. No. 5,192,326
U.S. Pat. No. 5,290,763
U.S. Pat. No. 5,332,475
U.S. Pat. No. 5,371,191
U.S. Pat. No. 5,458,643
U.S. Pat. No. 5,514,180
U.S. Pat. No. 5,534,028
U.S. Pat. No. 5,563,124
U.S. Pat. No. 5,645,597
U.S. Pat. No. 5,674,295
U.S. Pat. No. 5,800,537
U.S. Pat. No. 5,800,549
U.S. Pat. No. 5,817,153
U.S. Pat. No. 5,824,093
U.S. Pat. No. 5,837,235
U.S. Pat. No. 5,854,397
U.S. Pat. No. 5,922,028
U.S. Pat. No. 5,964,807
U.S. Pat. No. 5,976,186
U.S. Pat. No. 6,022,376
U.S. patent application Ser. No. 09/545,441.
U.S. patent application Ser. No. 09/746,921.
Pathak et al., “Bioerodible Hydrogels Based on Photopolymerized Poly(Ethylene Glycol)-Co-Poly(Alpha-Hydroxy Acid) Diacrylate Macromers,” Macromolecules, 26:581-87, 1993.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

1. A composition comprising nucleus pulposus tissue and at least a first cross-linkable viscosity control agent.

2-43. (canceled)

44. The composition according to claim 1 wherein at least a portion of the nucleus pulposus tissue is cross-linked, decellularized, denatured or rendered substantially non-immunogenic.

45. The composition according to claim 1, wherein the nucleus pulposus tissue comprises human tissue.

46. The composition according to claim 1, wherein the cross-linkable viscosity control agent is cross-linkable in situ.

47. The composition according to claim 1, wherein the cross-linkable viscosity control agent is cross-linkable by exposure to light.

48. The composition according to claim 1, wherein the cross-linkable viscosity control agent comprises a cross-linkable proteoglycan.

49. The composition according to claim 1, wherein the cross-linkable viscosity control agent is functionalized by the addition of at least a first cross-linkable moiety.

50. The composition according to claim 49, wherein the cross-linkable viscosity control agent comprises functionalized hyaluronic acid.

51. The composition according to claim 49, wherein the cross-linkable viscosity control agent is functionalized with glycidyl methacrylate.

52. The composition according to claim 49, wherein the cross-linkable viscosity control agent comprises at least a second distinct cross-linkable moiety.

53. The composition according to claim 52, wherein said second distinct cross-linkable moiety is vinyl pyrrolidinone.

54. The composition according to claim 1, wherein the concentration of the nucleus pulposus tissue is less than or equal to about 5% (wt./vol.).

55. A composition comprising nucleus pulposus tissue, at least a portion of which is cross-linked, and at least a first cross-linkable viscosity control agent.

56. The composition according to claim 55, wherein at least a portion of the nucleus pulposus tissue is decellularized, denatured or rendered substantially non-immunogenic.

57. The composition according to claim 55, wherein the nucleus pulposus tissue comprises human tissue.

58. The composition according to claim 55, wherein the cross-linkable viscosity control agent is cross-linkable in situ.

59. The composition according to claim 55, wherein the cross-linkable viscosity control agent is cross-linkable by exposure to light.

60. The composition according to claim 55, wherein the cross-linkable viscosity control agent comprises a cross-linkable proteoglycan.

61. The composition according to claim 55, wherein the cross-linkable viscosity control agent is functionalized by the addition of at least a first cross-linkable moiety.

62. The composition according to claim 61, wherein the cross-linkable viscosity control agent comprises functionalized hyaluronic acid.

63. The composition according to claim 61, wherein the cross-linkable viscosity control agent is functionalized with glycidyl methacrylate.

64. The composition according to claim 61, wherein the cross-linkable viscosity control agent comprises at least a second distinct cross-linkable moiety.

65. The composition according to claim 64, wherein said second distinct cross-linkable moiety is vinyl pyrrolidinone.

66. The composition according to claim 55, wherein the concentration of the nucleus pulposus tissue is less than or equal to about 5% (wt./vol.).

67. A composition comprising nucleus pulposus tissue, at least a portion of which is decellularized, and at least a first cross-linkable viscosity control agent.

68. The composition according to claim 67, wherein at least a portion of the nucleus pulposus tissue is cross-linked, denatured or rendered substantially non-immunogenic.

69. The composition according to claim 67, wherein the nucleus pulposus tissue comprises human tissue.

70. The composition according to claim 67, wherein the cross-linkable viscosity control agent is cross-linkable in situ.

71. The composition according to claim 67, wherein the cross-linkable viscosity control agent is cross-linkable by exposure to light.

72. The composition according to claim 67, wherein the cross-linkable viscosity control agent comprises a cross-linkable proteoglycan.

73. The composition according to claim 67, wherein the cross-linkable viscosity control agent is functionalized by the addition of at least a first cross-linkable moiety.

74. The composition according to claim 73, wherein the cross-linkable viscosity control agent comprises functionalized hyaluronic acid.

75. The composition according to claim 73, wherein the cross-linkable viscosity control agent is functionalized with glycidyl methacrylate.

76. The composition according to claim 73, wherein the cross-linkable viscosity control agent comprises at least a second distinct cross-linkable moiety.

77. The composition according to claim 76, wherein said second distinct cross-linkable moiety is vinyl pyrrolidinone.

78. The composition according to claim 67, wherein the concentration of the nucleus pulposus tissue is less than or equal to about 5% (wt./vol.).
